SEQUENCE LISTING (17 sequences)

SEQ ID NO: 1 - protein, 9 aa, Homo sapiens
PREEQYNST

SEQ ID NO: 2 - protein, 9 aa, Homo sapiens
PREEQFNST

SEQ ID NO: 3 - protein, 9 aa, Mus musculus
PREEQFNST

SEQ ID NO: 4 - protein, 9 aa, Mus musculus
PREAQYNST

SEQ ID NO: 5 - protein, 9 aa, Oryctolagus cuniculus
LREQQFNST

SEQ ID NO: 6 - protein, 9 aa, Rattus norvegicus
PREEQYNST

SEQ ID NO: 7 - protein, 9 aa, Rattus norvegicus
APEKQSNST

SEQ ID NO: 8 - protein, 9 aa, Bos taurus
PREEQFNST

SEQ ID NO: 9 - protein, 9 aa, Felis catus
PREEQFNST

SEQ ID NO: 10 - protein, 9 aa, Canis lupus
SREQQFNGT

SEQ ID NO: 11 - protein, 9 aa, Equus caballus
PKEEQFNST

SEQ ID NO: 12 - protein, 9 aa, Equus caballus
QREAQFNST

SEQ ID NO: 13 - protein, 9 aa, Equus caballus
PNEEQNNST

SEQ ID NO: 14 - protein, 9 aa, Equus caballus
PKQEQFNST

SEQ ID NO: 15 - protein, 9 aa, Equus caballus
PKEEQFNST

SEQ ID NO: 16 - protein, 9 aa, Equus caballus
AKEKQDNST

SEQ ID NO: 17 - protein, 9 aa, Equus caballus
PKQEQNNST

(19) United States
(12) Patent Application Publication
(10) Pub. No.: US 2024/0182586 A1
(43) Pub. Date: Jun. 6, 2024

(54) Site-Specific Modification of Glycoproteins Through Transglutaminase-Mediated Conjugation

(71) Applicant: Northeastern University, Boston, MA (US)

(72) Inventors: Zhaohui Sunny ZHOU, Wellesley, MA (US); Amissi SADIKI, Boston, MA (US)

(21) Appl. No.: 18/287,539

(22) PCT Filed: May 11, 2022
(86) PCT No.: PCT/US2022/028859

§ 371 Date: Oct. 19, 2023

Related U.S. Application Data
(60) Provisional application No. 63/187,376, filed on May 11, 2021.

(51) Int. Cl. C07K 16/28; A61K 39/00; C07K 1/107; C12N 9/10; C12N 9/24

(52) U.S. Cl. CPC C07K 16/2863; C07K 1/107; C12N 9/1044; C12N 9/1051; C12N 9/2402; A61K 2039/505; A61K 2123/00; C07K 2317/732; C07K 2317/90; C12Y 204/01133; C12Y 302/01096; C12Y 302/01097

(57) ABSTRACT

Methods for making a site-specific modification of a glycoprotein are provided which preserve the charge and glycoform of the glycoprotein. One or more glycans are trimmed from the glycoprotein, while keeping a core glycan attached to the glycoprotein. The removal of the one or more glycans provides access to a targeted amino acid for a site-specific reaction. The attached core glycan can optionally be utilized for modulation of the glycoprotein, or one or more glycans or reagents can be attached to the core.

### CROSS-REFERENCE TO RELATED APPLICATIONS

This application claims priority to U.S. Provisional Application No. 63/187,376, filed 11 May 2021, which is hereby incorporated by reference in its entirety.

### BACKGROUND

Non-specific chemical methods, such as acylation of amines (e.g., lysine) or alkylation of thiols (e.g., cysteine), are widely used to construct protein conjugates for many applications. These techniques have several shortcomings. For example, the lack of site-specificity results in heterogenous products, as well as a synthetic route that is irreproducible and prone to side reactions. Glycoproteins are particularly sensitive to non-specific modifications because of the added complexity of the oligosaccharide chains (glycans) attached to amino acid sidechains of the protein. Site-specific conjugation of antibodies is reviewed by Sadiki et al., 2020. Unpredictable modifications at or near the functional domains of glycoproteins may significantly reduce the targeting activity and specificity of the glycoproteins. Changes is binding specificity of antibodies are described by Cunningham et al., 2021. Genetic engineering of glycoproteins can take a bottom-up approach by altering the primary sequence to provide target sites for site-specific modification. This genetic engineering approach has various shortcomings, such as laborious optimization and low yield, provides only a linear architecture, and does not retain the native primary sequence and conformation. The protein conformation can primarily determine the site occupancy of glycosylated residues. To support the glycoprotein conformation, genetic engineering also can take a top-down or combined approach by, for example, transfecting non-human mammalian cells to modify a recombinant or native glycoprotein using human glycosyltransferases. The transfection approach may provide a high yield, but requires laborious front end optimization and testing because the products are unpredictable. Methods are needed to construct glycoprotein conjugates that better preserve the function of the glycoprotein while providing site-specific modifications.

### SUMMARY

The present technology provides methods for making a site-specific modification of a glycoprotein. The methods can preserve the charge and glycoform of the glycoprotein. A targeted amino acid for a site-specific modification is accessed by trimming one or more glycans from the glycoprotein, while keeping a core glycan attached to the glycoprotein. The attached core glycan can optionally be utilized for modulation of the glycoprotein or can be reattached to one or more glycans or reagents for glycan engineering.

The technology can be further summarized by the following list of features.

1. A method for making a site-specific modification of a glycoprotein, the method comprising the steps of:
(a) trimming a glycan linked to a glycosylation site amino acid residue of the glycoprotein, whereby a core of the glycan remains linked to the glycosylation site amino acid residue, and whereby a modification site amino acid residue of the glycoprotein becomes accessible for a reaction; and
(b) conjugating a reagent to the modification site amino acid residue made accessible in step (a).
   2. The method of feature 1, wherein the glycosylation site amino acid residue is an asparagine residue, and the glycan is an N-glycan linked to a side chain of the asparagine residue.
   3. The method of feature 1 or 2, wherein the modification site amino acid residue made accessible is a glutamine residue.
   4. The method of feature 3, wherein said glutamine residue is a naturally occurring glutamine residue or a non-naturally occurring glutamine residue for said glycoprotein.
   5. The method of feature 3 or 4, wherein the glycosylation site amino acid residue is an asparagine residue and the glutamine residue is adjacent to the asparagine residue or two residues away from the asparagine residue.
   6. The method of any of the preceding features, wherein the conjugation is catalyzed by a transglutaminase enzyme and the reagent is an amine-containing reagent.
   7. The method of feature 6, wherein the amine-containing reagent becomes covalently linked through its amine function to a side chain of the glutamine residue.
   8. The method of feature 6 or 7, wherein the transglutaminase enzyme is a naturally occurring transglutaminase, mutated transglutaminase, engineered transglutaminase, recombinant transglutaminase, micro-organism transglutaminase, mammalian transglutaminase, or protein-glutamine γ-glutamyltransferase E.C. 2.3.2.13.
   9. The method of any of the preceding features, wherein the glycoprotein comprises an N-linked glycosylation site having an amino acid sequence selected from the group consisting of SEQ ID NOS:1-17.
   10. The method of any of features 2-9, wherein the core of the N-glycan remaining after step (a) comprises at least one N-acetyl glucosamine residue.
   11. The method of feature 1, wherein the glycosylation site amino acid residue is a serine residue or a threonine residue, and the glycan is an O-glycan linked to a side chain of the serine or threonine residue.
   12. The method of feature 11, further comprising the step of, before or during step (a):
(a0) removing one or more sialic acid residues from the O-glycan using a sialidase.
   13. The method of any of the preceding features, wherein the trimming is performed using an endoglycosidase.
   14. The method of feature 13, wherein the endoglycosidase is a naturally occurring, mutated, recombinant, or engineered endoglycosidase.
   15. The method of feature 13, wherein the endoglycosidase is selected from the group consisting of Endo S, Endo S2, Endo H, Endo F1, Endo F2, Endo F3, Endo D, and Endo M
   16. The method of feature 13, wherein the endoglycosidase mediates hydrolysis of an N,N′diacetyl chitobiose bond.
   17. The method of feature 13, wherein the endoglycosidase is endo-α-N-acetylgalactosaminidase.
   18. The method of any of features 1-12, wherein the trimming is performed using a chemical method.
   19. The method of feature 18 comprising incubation of the glycoprotein with trifluoromethanesulfonic acid.
   20. The method of any of the preceding features, wherein said glycosylation site amino acid residue is neutrally charged before performing the method, and wherein the amino acid residue remains neutrally charged after performing the method.
   21. The method of any of the preceding features, further comprising at least one of the following additional steps performed before, during, or after step (b):
transferring a galactose moiety comprising a chemical handle onto the trimmed core glycan using a β-1,4-galactosyltransferase;
transferring an unnatural glycan substrate onto the trimmed core glycan using a glycosynthase;
removing a fucose from the trimmed core glycan using a fucosidase.
   22. The method of feature 21, wherein the glycan is an N-glycan, and the trimmed core of the N-glycan is a trimmed core N-glycan.
   23. The method of feature 22, wherein a galactose moiety comprising a chemical handle is transferred onto the trimmed core N-glycan using a galactosyltransferase, and wherein the method further comprises the step of:
attaching a functional group to the chemical handle; wherein the functional group comprises a chromophore, fluorophore, affinity tag or targeting agent, chelator, radioisotope, dye or contrast agent, ultrasound agent, targeting moiety, polyethylene glycol, or polymer.
   24. The method of any of the preceding features, wherein in step (b) the reagent comprises an amine-containing reagent that becomes covalently linked via an isopeptidic bond between an amine group of the amine-containing reagent and an acyl group on a side chain of the at least one of said one or more modification site amino acid residues.
   25. The method of any of the preceding features, wherein the reagent is an amine-containing reagent comprising a clickable handle for click chemistry.
   26. The method of feature 25, wherein the clickable handle comprises an azide functional group.
   27. The method of feature 26, further comprising the step of:
attaching a moiety comprising a complementary clickable handle, wherein the complementary clickable handle is capable of forming a bond between the azide functional group and the complementary clickable handle.
   28. The method of feature 27, wherein the complementary clickable handle comprises a strained alkyne functional group.
   29. The method of feature 28, wherein the complementary clickable handle forms a bond with the azide functional group via a strain promoted azide-alkyne cycloaddition (SPAAC) reaction.
   30. The method of any of the preceding features, wherein the reagent or the moiety comprising a clickable handle comprises a targeting moiety, an imaging moiety, an immunomodulator, a gene delivery vehicle, a therapeutic agent, a diagnostic agent, or a polymer.
   31. The method of any of the preceding features, wherein the reagent is an amine-containing reagent comprising a stimulus-responsive linker selected from the group consisting of a photo-responsive linker, acid-cleavable linker, reducible linker, peptide or dipeptide linker, and a ß-glucuronide linker.
   32. The method of any of the preceding features, wherein the glycoprotein is selected from the group consisting of antibodies or antibody fragments and glycosylated enzymes.
   33. The method of any of the preceding features, wherein the glycoprotein is a naturally occurring or non-naturally occurring antibody or antibody fragment.
   34. The method of any of the preceding features, wherein the glycoprotein has a biological activity which is preserved after said site-specific modification.
   35. The method of any of the preceding features, wherein a naturally occurring antibody is modified while retaining its primary sequence, core glycan, and binding specificity.
   36. A modified glycoprotein produced by the method of any of the preceding features.
   37. A kit for site-specific modification of a glycoprotein, the kit comprising:
an endoglycosidase;
a transglutaminase; and
instructions for performing the method of any features 1-35.
   38. The kit of feature 37, further comprising a galactosyltransferase or a glycosynthase.
   39. The kit of feature 37 or 38, further comprising an amine-containing reagent.
   40. The kit of feature 39, wherein the amine-containing reagent comprises a targeting moiety, an imaging moiety, an immunomodulator, a gene delivery vehicle, a therapeutic agent, a diagnostic agent, or a polymer.
   41. The kit of feature 39 or feature 40, wherein the amine-containing reagent comprises an azide functional group that can undergo biorthagonal conjugation, such as inverse electron demand Diels-Alder (IEDDA) involving 1,2,4,5-tetrazine and an olefin.
   42. The kit of feature 39 or feature 40, comprising a moiety comprising a strained alkyne functional group and instructions for performing a SPAAC reaction.
   43. A method for modulating an effector function of an antibody, the method comprising the steps of:
(a) trimming a glycan linked to a glycosylation site amino acid residue of the antibody using an endoglycosidase, whereby a core of the glycan remains linked to the glycosylation site amino acid residue, and whereby the effector function of the antibody is reduced or abolished; and
(b) transferring a glycan substrate onto the core of the glycan using a glycosynthase or removing a fucose from the core of the glycan using a fucosidase, whereby the effector function is at least partially recovered.
   44. The method of feature 43, wherein the endoglycosidase is Endo S2.
   45. The method of feature 43 wherein the effector function is abolished after performing step (a).
   46. The method of any of features 43-45, wherein the effector function is binding of an Fc domain of the antibody to an Fcγ receptor on an immune cell.
   47. The method of any of features 43-46, wherein the effector function comprises antibody-dependent cellular cytotoxicity or complement-dependent cytotoxicity.
   48. The method of any of features 43-47, wherein the glycan substrate comprises oxazoline.
   49. The method of any of features 43-48, wherein the glycosylation site amino acid residue is an asparagine residue, and the glycan is an N-glycan linked to a side chain of the asparagine residue.
   50. The method of feature 43, wherein the antibody comprises one or more glutamine residues, and
wherein in step (a) the one or more glutamine residues become accessible to a transglutaminase enzyme for a transglutaminase-catalyzed reaction between the one or more glutamine residues and an amine-containing reagent.
   51. The method of feature 50, wherein said one or more glutamine residues are naturally occurring glutamine residues or non-naturally occurring glutamine residues for said antibody.
   52. The method of feature 50 or feature 51, further comprising the step of, before, during, or after step (b):
(b1) conjugating the amine-containing reagent to the one or more accessible glutamine residues via the transglutaminase-catalyzed reaction, whereby the reagent becomes covalently linked through its amine function to a side chain of at least one of said one or more glutamine residues.
   53. An antibody having a modulated effector function, the antibody made by the method of any one of features 43-52.
   54. The modified glycoprotein of feature 36 or the modulated antibody of feature 53 that is suitable for use as an imaging agent.
   55. The modified glycoprotein or the modulated antibody of feature 54, wherein the imaging is of a cell or of a tumor.
   56. The modified glycoprotein of feature 36 or the antibody having a modulated effector function of feature 53 that is suitable for use in a targeted therapy for treating a cancer, for use in a biological assay, for use in a diagnostic procedure, or for use in a method comprising immunotherapy.

As used herein, the term “about” refers to a range of within plus or minus 10%, 5%, 1%, or 0.5% of the stated value.

As used herein, “consisting essentially of” allows the inclusion of materials or steps that do not materially affect the basic and novel characteristics of the claim. Any recitation herein of the term “comprising”, particularly in a description of components of a composition or in a description of elements of a device, can be exchanged with the alternative expression “consisting of” or “consisting essentially of”.

### BRIEF DESCRIPTION OF THE DRAWINGS

FIG. 1A at top shows an illustration of a transglutaminase (TGase) catalyzed modification of a glutamine (Gln295) of an antibody using previously reported work that requires complete removal of the N-linked glycan by an amidase (PNGase F) and conversion of asparagine (Asn297) to aspartic acid (Asp297). An example of the present technology is shown at the bottom of FIG. 1A; trimming of the N-linked glycan's innermost N-acetylglucosamine (GlcNAc; highlighted) on Asn297 using endoglycosidase (EndoS2) makes the Gln295 accessible to TGase catalyzed modification without requiring conversion of Asn297 to Asp297. FIG. 1B shows a schematic illustration of a method for site-specific modification of a glycoprotein according to the present technology.

FIG. 2A and FIG. 2B show perspective views of crystal structures of human antibody Fc fragment (PDB 4CDH) highlighting the PNGase F and EndoS2-mediated cleavage sites on Asn297 at the sidechain's amide bond and at the innermost GlcNAc, respectively. FIG. 2C shows a zoomed-out view of a crystal structure of human antibody (PDB 1HZH) illustrating the position of the Fc glycans. Images are rendered in Pymol version 2.4.

FIG. 3 shows a comparison of the effect of PNGase F and EndoS mediated glycan removal on the isoelectric point of cetuximab using an isoelectric focusing gel and Coomassie staining.

FIG. 4A illustrates antibodies conjugated at Gln sidechain with an amine-containing, azide or alkyne terminated clickable handle (lin) are further derivatized with a strained alkyne or azide terminated payload (ker) via click chemistry to form the customizable linker at right. FIG. 4B shows examples of clickable handles and strained alkynes for assembly of antibody conjugates via strain-promoted azide-alkyne cycloaddition click chemistry.

FIG. 5A and FIG. 5B illustrate site-specific TGase-mediated conjugation of polyethylene glycol (PEG) and chromophore (fluorophore) onto cetuximab. FIG. 5A shows an SDS-PAGE analysis (Coomassie stain) with near quantitative installing of a 10 kDa PEG groups onto the heavy chain (HC) of the antibody (lanes 3 and 4). FIG. 5B shows fluorescence imaging of an SDS-PAGE analysis of antibody-fluorophore conjugates with modification primarily at the heavy chain (HC) of the antibody.

FIG. 6 shows SDS-PAGE of cetuximab-PEGylated conjugates with Coomassie staining under native and reducing conditions.

FIG. 7 and FIG. 8 show reducing SDS-PAGE of cetuximab-chromophore conjugates with fluorescence imaging (excitation 608-632 nm and emission 675-720 nm) at top and Coomassie staining at bottom.

FIG. 9 shows reducing SDS-PAGE of infliximab-chromophore conjugates.

FIG. 10 shows nonreducing SDS-PAGE of cetuximab-chromophore conjugates.

FIG. 11 shows reducing SDS-PAGE of infliximab-chromophore conjugates.

FIG. 12 shows reducing SDS-PAGE of cetuximab-chromophore conjugates.

FIG. 13 shows nonreducing SDS-PAGE of cetuximab-chromophore conjugates.

FIG. 14A shows fluorescence confocal microscopy images of ovarian cancer cells (Ovcar3) stained with cetuximab fluorophore conjugates (Cet-AF647, left) or without Cet-AF647 (right). Both groups of cells (left and right) are stained with Hoechst 33342 nuclear stain. FIG. 14B shows a plot of fluorescence flow cytometry results from Ovcar3 cells stained with Cet-AF647 compared to no stain.

FIGS. 15A-15C show analyses of Ovcar3 cells via flow cytometry. FIG. 15A and FIG. 15B show exemplary forward scatter (FSC) and side scatter (SSC) dot plots of raw data (top) showing cell gating scheme and AF647 fluorescence histograms (bottom) for unstained cells (15A) and cells stained with Cet-AF647 (15B). FIG. 15C shows two exemplary overlay plots comparing unstained and stained cell populations. While acquiring the data, each group is sampled 6 times and mean fluorescence intensity is recorded for further analysis. Further analysis is shown in FIG. 14B.

FIG. 16A and FIG. 16B show results of a thermal shift stability assay of cetuximab (Cet), cetuximab treated with EndoS2 (Cet-EndoS2; also named trimmed cetuximab), cetuximab treated with PNGase F (Cet-PNGase F), and lastly, EndoS2-trimmed cetuximab incubated with mTGase, 2-azidoethanamine, and then 10 kDa DBCO-PEG-10 kDa (Cet-PEG). FIG. 16C shows a summary plot of melting temperatures (Tm) obtained from the thermal shift assay.

### DETAILED DESCRIPTION

The present technology provides methods for making a site-specific modification of a glycoprotein. The site-specific methods do not require changing the glycoform or conformation of the glycoprotein. The site-specific methods can preserve the effector functions of a glycoprotein after the modifications. For example, specificity of antibodies can be preserved after performing the methods. The methods can be used to modulate the functions of glycoproteins, for example, to decrease non-specific binding of labeled antibody-based imaging agents to fragment-gamma receptors (FcγRs) on immune cells.

Approximately half of proteins typically expressed in a cell undergo glycosylation, which entails the post translational covalent addition of sugar moieties (glycans) to specific amino acids. However, few methods exist to site-specifically conjugate reagents to glycoproteins, especially without the need for genetic engineering. Glycoproteins, such as antibodies, are of particular interest because these proteins largely control immune response.

An example technique is using transglutaminase (TGase, EC 2.3.2.13) to modify glutamine sidechains on glycoproteins. The TGase enzyme catalyzes the formation of an isopeptide amide bond between an unsubstituted. sidechain amide of glutamine residue (as an acyl donor) and a nucleophilic amine substrate (as an acyl acceptor). To catalyze the isopeptide bond, the TGase must have access to at least one glutamine sidechain of the glycoprotein. Glycans on the glycoprotein can sterically hinder all access to glutamine by TGase.

The usefulness of TGase for conjugating reagents to glycoproteins is severely limited by the steric hindrance. One approach is to cleave the glycans from a glycoprotein before conjugating a reagent to a glutamine sidechain of the glycoprotein. Complete removal of the glycans should be avoided because the glycans are key to the specific function, structure, and immunogenicity of the glycoprotein in vivo.

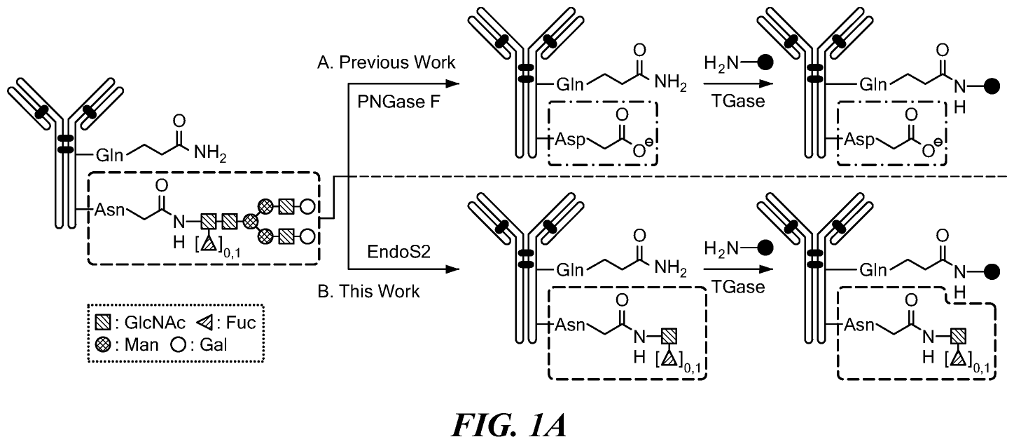

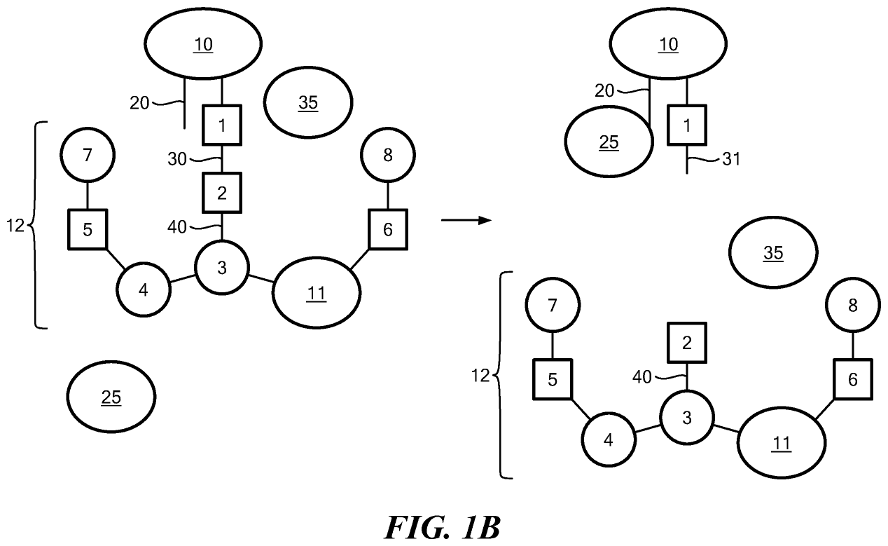

For targeted site-specific modification of a glycoprotein, access to a sidechain of a target modification site amino acid can be completely hindered by glycans covalently attached to nearby amino acid sidechains. At the left side of FIG. 1A, a glutamine sidechain (Gln-CH₂—CH₂—C═O—NH₂) is depicted as a neutrally charged, glycosylated asparagine with an N-glycan including an attached innermost core N-acetylglucosamine (GlcNAc) further attached to GlcNAc, mannose (Man) and galactose (Gal) carbohydrates. The Man and Gal hinder TGase access to the glutamine sidechain. At the top of FIG. 1A, previous work reports removing the N-glycan with an exoglycosidase (PNGase F) catalyzed reaction to convert the neutral asparagine (an amide) to a negatively charged aspartic acid (a carboxylic acid).

Despite having over 60 glutamine residues, native (non-modified) antibodies are poor substrates for TGase. In this context, native antibodies can be antibodies that are either recombinantly produced or naturally existing in various species whereby their primary amino acid sequence is conserved. The sequences of native antibodies, though, are largely identified and conserved, in particular the Fc regions. TGase can be applied to native antibodies after entire glycans have been cleaved from the native antibodies. Applied to IgG1, prior to transamidation (access to a glutamine residue by TGase), one has to reduce the steric hindrance from a nearby conserved N-glycan (e.g., asparagine, Asn297, IgG1). The removal of the steric hindrance by completely cleaving glycans from Asn297 is illustrated at the top of FIG. 1A.

In a previous report, an amidase (PNGase F, E.C. 3.5.1.52) is used to completely remove the N-glycan from Asn297 (Dennler, et al., 2014). During this removal, PNGase F also converts a net neutral asparagine (Asn297) to a negatively charged aspartic acid (Asp297, IgG1, FIG. 1A top). While removing the steric hindrance, this charge alteration can markedly change the conformation of the antibody. In the top of FIG. 1A, only a single conserved glutamine (Gln295, IgG1) in the Fc region of IgG is modified by TGase (e.g., TGase E.C. 2.3.2.13), thereby providing a highly specific and generally applicable conjugation method (Josten, et al., 2000; Mindt, et al., 2008; Jeger, et al., 2010).

The complete deglycosylation of Asn297, e.g., by PNGase F, has several disadvantages. First, deglycosylation transforms the Asn297 (an amide) into aspartic acid Asp297 (a carboxylic acid). This deamidation process results in a negatively charged group at physiological conditions. The charge variant leads to numerous issues such as significant structural changes, functional perturbation and immunogenicity, which ultimately may affect the function and efficacy of the antibody conjugates. This transformation results in a conformational change that may lead to decrease in stability in vivo, increase aggregation tendency, as well as abolishment of Fc-related biological activity such as effector function, for example, antibody dependent cellular cytotoxicity (ADCC).

Protein engineering is also reported and includes insertion or deletion of reactive glutamine(s) via mutagenesis or incorporation of peptide tags (with a reactive glutamine) onto the terminal ends of the antibody (Strop, et al., 2016; Anami, et al., 2017; Schneider, et al., 2020). These genetic engineering approaches have various shortcomings such as laborious optimization, low yield, provide only a linear architecture and do not retain the native primary sequence. Consequently, not all antibodies are amenable to the genetic route.

An example of the technology disclosed herein is illustrated at the bottom of FIG. 1A. An endoglycosidase (EndoS2) catalyzed reaction is used to trim glycans from the neutral asparagine and to make the glutamine sidechain accessible to TGase. After the trimming, the neutral asparagine is retained and also the core glycan attached to the asparagine. This construct is homogenous with a single glycoform and more likely to have reduced immunogenicity than the previously reported methods.

The example depicted at the bottom of FIG. 1A can be applied to antibodies such as Immunoglobulin G (IgG). Table 1 below shows the amino acid sequence alignment of sites of glutamine modification mediated by TGase. The glutamine modification sites are at the centered ‘Q’ of each sequence (Gln, Q; Q295, IgG1). The N-glycosylation sites (Asn, N; N297 for IgG1) are to the right of the centered Q in each. The sequences are aligned using EMBL-EBI Clustal Omega.

TABLE 1
Amino Acid Sequence Alignment

| Sequence name: | Sequence: | Source: |
|---|---|---|
| SEQ ID NO: 1 | PREEQYNST | human IgG 1 |
| SEQ ID NO: 2 | PREEQFNST | human IgG 2-4 |
| SEQ ID NO: 3 | PREEQFNST | mouse IgG 1 |
| SEQ ID NO: 4 | PREAQYNST | mouse IgG 3 |
| SEQ ID NO: 5 | LREQQFNST | rabbit IgG |
| SEQ ID NO: 6 | PREEQYNST | rat IgG 1 |
| SEQ ID NO: 7 | APEKQSNST | rat IgG 2a |
| SEQ ID NO: 8 | PREEQFNST | bovine IgG 1-2 |
| SEQ ID NO: 9 | PREEQFNST | feline IgG |
| SEQ ID NO: 10 | SREQQFNGT | canine IgG |
| SEQ ID NO: 11 | PKEEQFNST | equine IgG 1 |
| SEQ ID NO: 12 | QREAQFNST | equine IgG 2 |
| SEQ ID NO: 13 | PNEEQNNST | equine IgG 3 |
| SEQ ID NO: 14 | PKQEQFNST | equine IgG 4 |
| SEQ ID NO: 15 | PKEEQFNST | equine IgG 5 |
| SEQ ID NO: 16 | AKEKQDNST | equine IgG 6 |
| SEQ ID NO: 17 | PKQEQNNST | equine IgG 7 |

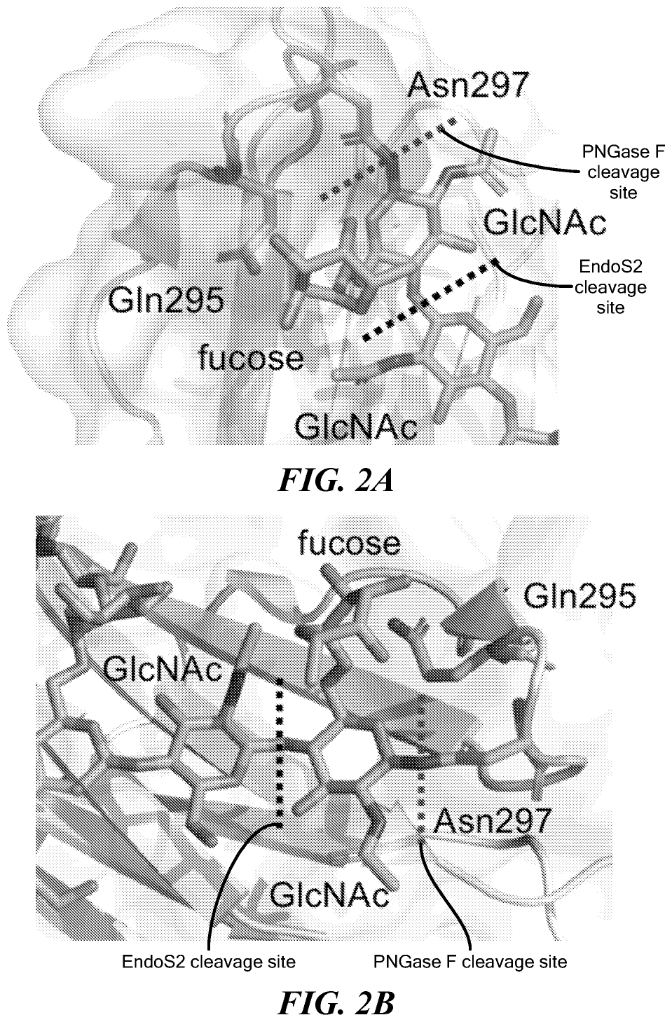

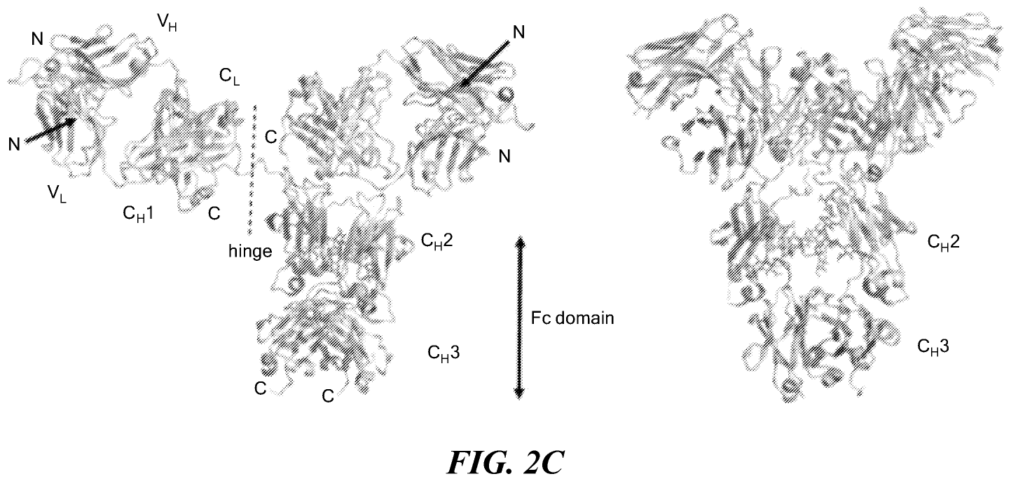

The methods herein enable tailoring of N-glycans, site specific-modification of the associated glycoprotein, and optional modulation of the glycoprotein. As depicted in FIG. 1A and FIGS. 2A-2B 1 and 2, endoglycosidase (e.g., EndoS, E.C. 3.2.1.96) trims the N-glycan to its core glycan N-acetylglucosamine (GlcNAc). EndoS are a family of hydrolases that selectively hydrolyze N-linked glycans in the Fc region of a native antibody and leaves the innermost GlcNAc intact (Collin & Olsen, 2001; Sjogren, et al., 2013). Unlike the amidase or glycosidase (PNGase F), EndoS-mediated hydrolysis maintains the neutral Asn297 as shown at bottom of FIG. 1A. This EndoS enzyme is commercially available and applicable to a range of antibody isotypes (e.g., human IgG 1, 2, 3 and 4), and thus has found utility in simplifying analysis, bioconjugation, diagnosis and increased specificity in imaging. FIG. 2C illustrates a zoomed-out crystal structure of human antibody (PDB 1HZH) illustrating the position of the Fc glycans. FIG. 2A and FIG. 2B show enlarged crystal structures of human antibody's Fc fragment (PDB 4CDH) highlighting the PNGase F and EndoS2-mediated cleavage sites on Asn297 at the side chain's amide bond and the innermost GlcNAc.

Naturally occurring or engineered enzymes such as Endoglycosidases (EndoS, E.C. 3.2.1.96) can be used to trim glycans on antibodies. Endo S is derived from Streptococcus pyogenes and includes a family of enzymes that catalyze a hydrolysis reaction of complex type N-linked glycans (e.g., innermost core N-Acetylglucosamine, GlcNAc) on macromolecules such as antibodies. EndoS exhibits endo-β-N-acetylglucosaminidase activity with catalytic glutamic residues and several tryptophan residues that play an important role in catalysis (Sjogren, et al., 2013). This enzyme displays a variety of substrate specificities. For example, EndoS hydrolyzes N-linked glycans in native antibodies and not denatured antibodies. Other examples of endoglycosidases include Endo S2, Endo H, Endo F1, Endo F2, Endo F3, Endo D, Endo M, O-glycosidase derived from Streptococcus oralis, O-glycosidase derived from Streptococcus pneumoniae, and O-glycosidase derived from Enterococcus faecalis (Fujita, et al., 2005; Koutsioulis, et al., 2008). EndoS is commercially available and applicable to a wide range of antibody isotypes including human IgGs 1-4, mouse IgGs 1 and 3, rabbit IgG, rat IgGs 1 and 2a, bovine IgGs 1 and 2, feline and canine IgGs, as well as equine IgGs 1-7 (e.g., Table 1). O-Glycosidase, also known as endo-α-N-acetylgalactosaminidase (EC 3.2.1.97), catalyzes the removal of Core 1 and Core 3 O-linked disaccharides from glycoproteins.

At the bottom of FIG. 1A, in a second step, TGase is used to introduce a desired amine-containing group to specific-site. TGase is also commercially available and applicable to a wide range of antibody isotypes (e.g., human IgG 1, 2, 3 and 4; murine IgG 1 and 3, and rat IgG1, 2a, 2b and 2c). Examples of TGase include naturally occurring transglutaminase, mutated transglutaminase, engineered transglutaminase, recombinant transglutaminase, micro-organism derived transglutaminase, mammalian transglutaminase, and protein-glutamine γ-glutamyltransferase E.C. 2.3.2.13.

The technology provides new methods for modification of antibodies and their related fragments via a chemoenzymatic process. The method includes trimming of native or engineered glycan(s) on antibodies and their related fragments, namely glycan remodeling, to render antibody's glutamine residue(s) accessible for conjugation by TGase. This can be followed by conjugation of a native or engineered glutamine residue(s) with an amine containing reagents or other suitable reagents via a transamidation reaction mediated by TGase.

The methods presented herein are proven to provide advantageous attributes. First, the endoglycosidase-hydrolysis (i.e., glycan remodeling or trimming) results in a single and homogenous glycoform. This construct can be less immunogenic than the previous PNGaseF hydrolysis that converts a conserved asparagine to an aspartic acid. Second, the glycan remodeling process is versatile by facile modulation of Fc-related biological activity, e.g., ADCC. Model antibodies are utilized herein. Both model antibodies utilized (cetuximab and Infliximab) have effector function. To completely abolish the effector function, EndoS2 can be utilized. The abolishment of the effector function results in better imaging agents by decreasing non-specific binding; for example, by decreasing binding of the Fc domain to Fcγ receptors on immune cells.

A facile process to build antibody-chromophore conjugates via convergent assembly is provided. This approach confers a higher binding efficiency than its non-specific conjugation equivalent. A new method to modify a native antibody using TGase while retaining the antibody's primary sequence and core glycan is also provided.

The technology can be applied to a variety of glycoproteins. The endoglycosidase can be used to trim an O-glycan linked to a sidechain of the serine or threonine residue. Trimming of the O-glycan causes a modification site amino acid residue to become accessible for attachment of a reagent. Sialic acids can be removed from the O-glycan using a sialidase.

The technology contemplates that as more endoglycosidases become available, the methods can be applied to a larger variety of glycoproteins. An schematic diagram of an embodiment of a method for making a site-specific modification of a glycoprotein is shown in FIG. 1B. Glycoprotein 10 is depicted at left with the side chain of modification site amino acid residue 20 sterically hindered by glycan 12. The glycoprotein has an attached core glycan 1, which is attached to a second glycan portion 2 by bond 30. The second glycan portion is further attached to third glycan portion 3 by bond 40. The third glycan portion is attached to extending glycan portions 4, 5, and 7 on one side of the glycan. The third portion is attached to extending glycan portions 11, 6, and 8 on the other side of the glycan. Without trimming, reagent 25 cannot access the modification site amino acid residue due to steric hindrance. Endoglycosidase 35 is used to catalyze trimming at bond 30 or at bond 40. Glycan portion 13 is cleaved off by the endoglycosidase, resulting in removal of steric hindrance to the modification site amino acid residue 20. After the endoglycosidase cleavage, only the core glycan remains linked to the glycoprotein. Reagent 25 can then access the modification site amino acid residue 20. Attachment site 31 can be utilized for attachment of additional reagents, or reagent 25 can be attached at site 31. Optionally, glycan portion 13 can be reattached at site 31.

Trimming of an N-glycan or an O-glycan can also be performed using a chemical method instead of an endoglycosidase. An example of such a chemical method is reported by Sojar et al. (1987). That method uses incubation of the glycoprotein with trifluoromethanesulfonic acid at 0° C. for 0.5 to 2 hours followed by neutralization with aqueous pyridins at −20° C.

A target modification site amino acid of the glycoprotein is not accessible due to steric hindrance of glycans on one or more nearby glycosylation site amino acids. After trimming of an N-glycan or an O-glycan, a sidechain of the target modification site amino acid residue of the glycoprotein becomes accessible. Depending on the size and characteristic of the steric hindrance, the modification site amino acid residue can be adjacent to the glycosylation site amino acid, 2 residues away from the glycosylation site amino acid, 3 residues away from the glycosylation site amino acid, 4 residues away from the glycosylation site amino acid, or 5 residues away from the glycosylation site amino acid. In another example, the nearby glycosylation site amino acid can be greater than 5 residues away from the modification site amino acid but may be in proximity to the modification site due to secondary (e.g., folding), tertiary, or quaternary structure of the glycoprotein.

Depending on the size of the glycans on the glycosylation site amino acid(s), the sidechain of the modification site amino acid residue can be about 1 nm away from the steric hindrance caused by glycans attached to the glycosylation site amino acid, about 2 nm away from the steric hindrance, about 3 nm away from the steric hindrance, about 4 nm away from the steric hindrance, of about 5 nm away from the steric hindrance. In another example, the sidechain of the modification site amino acid residue is greater than 5 nm away from the steric hindrance due to reagent or catalyst size.

Attachment of a reagent to an accessible modification site amino acid residue can be done by direct attachment of the reagent or by first attaching one or more linkers to the modification site amino acid residue. Attachment of the reagent can be accomplished by enzymatic or chemical methods. Examples of protein labeling reagents are compounds with reactive groups that facilitate covalent binding with proteins. N-hydroxy succinimide esters, for example, will attach to amino groups in proteins, such as lysine residues. Maleimide reactive groups selectively react with protein residues containing a sulfhydryl group, such as cysteines. Conjugates with hydrazide reactive groups can attach via carbonyl groups. Reagents are available for the attachment of conjugates, dyes, and other moieties to proteins. These labeling reagents, which can proceed with a simple reaction and purification step, can be useful in attaching conjugates such as gold, biotin, fluorophores, and dyes to purified proteins and antibodies. Example downstream applications of labeled proteins can include immunoassays, Western blots, and immunohistochemistry.

In another example, after trimming of an N-glycan or an O-glycan, a reagent or a linker can be attached to the core glycan on the trimmed N-glycan or O-glycan. The reagent or linker can be further attached or bridged to the modification site amino acid residue. Upon hydrolysis or trimming of the glycans attached to a glycoprotein, multiple enzymes such as TGase and galactosyltransferases can be used to construct dual and multipurpose macromolecule conjugates (as illustrated in FIG. 2A and FIG. 2B). β-1,4-galactosyltransferase (GalT, EC 2.4.1.38) is a family of enzymes that catalyze a transfer of an unnatural sugar moiety (e.g., galactose) with various chemical handles (e.g., azide or ketone) into glycans on macromolecules (e.g., antibodies) in the presence of a co-factor (Mn2+ ion). Once these handles are incorporated, further derivatization of a diverse array of functional group such as fluorophores, affinity tags, chelators, or custom chemistries can be enabled.

The technology provides further derivatization of glycoproteins at two or more sites. A wide array of functionalization can be achieved by TGase-mediated bioconjugation. Various amine substrates can be attached including heterobifunctional, branched, noncanonical and proteolytically cleavable. Moreover, both endogenous and exogenous stimuli-responsive linkers can be incorporated, e.g., to create photo-responsive molecules (Moulton, et al., 2019). Since TGase bioconjugation is orthogonal to other approaches, multi-functionalization can be achieved by a combination of TGase and other chemo-enzymatic tools such as galactosyltransferases or glycosynthases (Scallon, et al., 1995; Tsai, et al., 2017; Manabe, et al., 2019). New and creative conjugates are envisioned that combine distinct modalities, e.g., protein-antibody conjugates or virus-antibody conjugates (Park, et al., 2020).

The technology can provide design of the glycoprotein entity, such as conjugation at a single or dual site(s) on an antibody. Methods and reagents to trim a glycan of a glycoprotein and their related fragments (i.e., glycan remodeling) using an endoglycosidase enzyme are provided. By remodeling the core glycan, the methods and reagents can be used to modulate effector function, e.g., ADCC or complement-dependent cytotoxicity.

The methods disclosed herein maintain core glycans in glycoproteins and in antibodies. No generation of charge variants is required. Multi-functionalized glycoproteins can be readily produced via attachment at two sites: i.e., glycan and glutamine residues. The technology can be utilized to construct antibody drug conjugates, protein drugs, PEGylated drugs.

Prior to the technology disclosed herein, it was unpredictable whether a trimmed glycan would allow a reagent or a TGase catalyzed reaction to access a modification site amino acid residue, and if so, how efficient an attempted modification would be. In an example, near quantitative conversions are observed for both steps (trimming and TGase) and for multiple antibodies. The results suggest that other glycan trimming enzymes, such as EndoF1, EndoD and EndoH, can be equally successful. Further conjugation via the remaining glycans may be used, as demonstrated by GlyCLICK (Genovis), in which saccharides can be installed onto the glycans, e.g., for glycan remodeling and/or installation of additional bioconjugation handles.

The methods can modulate Fc-related biological activity. The site-specific methods can be used to modulate the functions of glycoproteins, for example, to decrease non-specific binding of labeled antibody-based imaging agents to fragment-gamma receptors (FcγRs) on immune cells (Gao, P., et al., 2015). The resulting conjugates using this methodology are likely to have abolished or markedly reduce effector function. For imaging applications, this feature is useful by decreasing nonspecific binding such as reducing binding of the Fc domain to Fcγ receptors on immune cells.

Conversely, to enhance effector activity, unnatural glycan substrates such as oxazolines can be reintroduced, after trimming a glycan, using a glycosynthase and/or removal of fucose using fucosidases to recover the effector function. The glycan remodeling process enables fine-tuning of the glycans and thus facile modulation of Fc-related biological activity, e.g., ADCC that are mediated by Fc receptors. Both model antibodies utilized (cetuximab and Infliximab) have effector function. To completely abolish this activity, EndoS can be utilized. Conversely, to enhance this activity, unnatural glycan substrates (such as oxazolines) can be re-introduced using a glycosynthase and/or removal of fucose using fucosidases to recover the effector function.

A novel method is presented to modify antibodies that has surprising advantages. First, it does not require complete removal of Fc domain's N-glycan. Second, unlike the previous procedures, the resulting antibody does not generate a charge variant, while maintaining the primary sequence (i.e., native form of the antibody) and core glycan (GlcNAc). Overall, the products are homogenous with only a single glycoform, well-defined, efficacious and more likely to have reduced immunogenicity and greater stability than the previous methods.

Trimmed glycans on antibodies render glutamine residue(s) accessible for transamidation reaction, e.g., mediated by TGase. Transglutaminase (TGase, e.g., E.C. 2.3.2.13) is a family of enzymes that catalyzes acyl transfer reactions of the unsubstituted amide in glutamine (Gln or Q) sidechain in proteins via a thioester intermediate (see FIG. 2A and FIG. 2B). Three common reactions include (1) cross-linking of glutamines with lysine (sidechain amine) in peptides/proteins or amines in peptides or polymers, (2) with primary amines to form substituted amides (e.g., bioconjugation), as well as (3) deamidation of glutamine through the hydrolysis of the thioester intermediate. All TGases use a catalytic cysteine residue; and many generally involve the classic catalytic triad: e.g., cysteine, histidine, and aspartic acid in the microbial TGase from Streptomyces mobaraensis.

A simple, robust and adaptable system is presented to generate photoremovable protein conjugates. The utility is validated by generation of a photoactivable protein, E. coli polymerase manager UmuD. These dynamic switches in proteins that impart spatial and temporal control are valuable to manipulate biological systems. An approach is presented to label glutamines with an ¹⁵N isotope in native peptides and proteins. This process obliviates the need for metabolic labeling and/or recombinant production, which are not readily accessible to many proteins and proteoforms. See U.S. Pat. No. 11,129,790B2, Chemo-Enzymatic Site-Specific Modification of Peptides and Proteins to Form Cleavable Conjugates.

Glycans play essential and critical roles in the structures and functions of numerous proteins. For immunoglobulins (IgG), a major function is a defense mechanism in humans against pathogens and foreign agents. For example, glycans are involved in humoral immune response through interactions between N-glycans on IgG with Fcγ receptors on immune cells to mediate effector function such as ADCC or complement-dependent cytotoxicity. Furthermore, variations in the glycosylation patterns may lead to pharmacodynamic, pharmacokinetic, and stability differences in protein pharmaceuticals, which influences the antibody's product quality, safety, and efficacy.

These methodologies can produce homogenous antibody conjugates and their related fragments for various applications such as analysis, imaging, radioimmunoconjugates, cytotoxicity using antibody-drug-conjugates (ADCs), PEGylation, and glycoengineering.

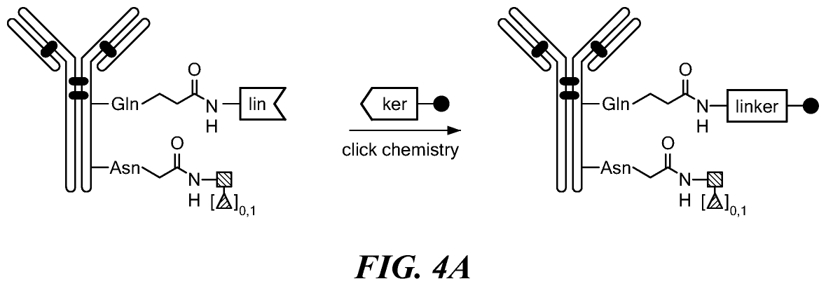

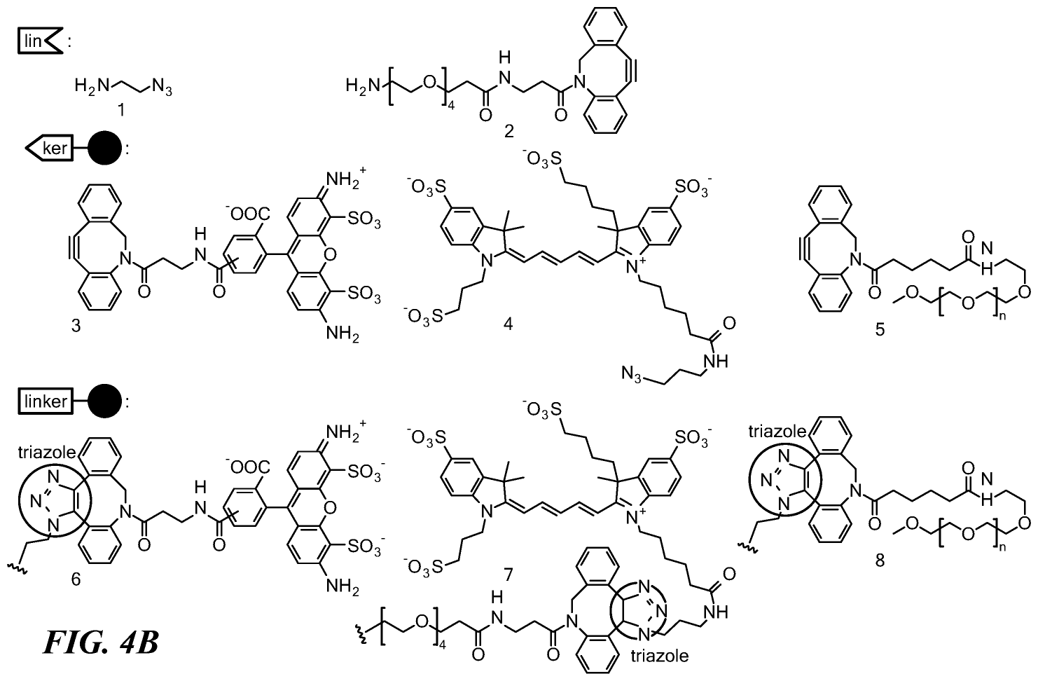

Surprisingly, all amine-containing molecules evaluated are excellent substrates for microbial transglutaminase (compounds 1 and 2; FIG. 4B) after EndoS mediated trimming of glycan in cetuximab. This surprise finding suggests that TGase can readily access the glutamine residue (Gln 295) after trimming of the innermost glycan. Various amine-containing substrates are evaluated using microbial transglutaminase (mTGase), which converts unsubstituted amides on glutamines into substituted amides. The use of mTGase has broad specificity towards amines with two main features: primary amines are generally accepted and substituents at the alpha position significantly slow the reaction down.

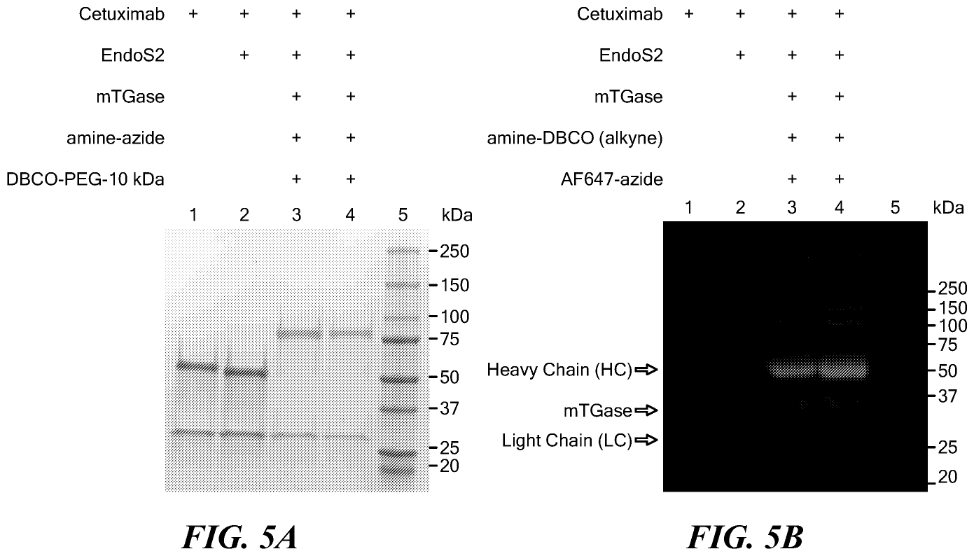

Modifications using compounds 1 and 2 in FIG. 4B as substrates for mTGase are confirmed by attachment of 10 kDa polyethylene glycol (PEG-10 kDa, compound 4, FIG. 4B) and various fluorophores (compounds 3 and 5) onto cetuximab via a two-step click chemistry reaction, namely strain-promoted azide-alkyne cycloaddition (SPAAC). Similarly, other click reactions can be utilized in a similar fashion such as inverse electron demand Diels-Alder (IEDDA, involving 1,2,4,5-tetrazine and olefins) or copper-catalyzed azide-alkyne cycloaddition (CuAAC). The SPAC reaction is quantitative, as demonstrated by a complete shift in the electrophoretic mobility of the antibody's heavy chain (HC) conjugated to a 10 kDa PEG (FIG. 5.A., lanes 3 and 4). The unmodified heavy chain (HC) of the antibody was not observed in FIG. 5A. Cetuximab contains complex biantennary Fc N-glycans that are highly heterogenous (both fucosylated and non-fucosylated exist) and most species are fucosylated (Qian, et al., 2007).

Since the overall yield is quantitative, it is shown that both fucosylated and non-fucosylated or glycan species are modified, which further expands the utility of the technology. The TGase-catalyzed transformation results in a modification at a single site (Gln295) in the heavy chain (HC) of the antibody (FIG. 5.B., lanes 3 and 4)

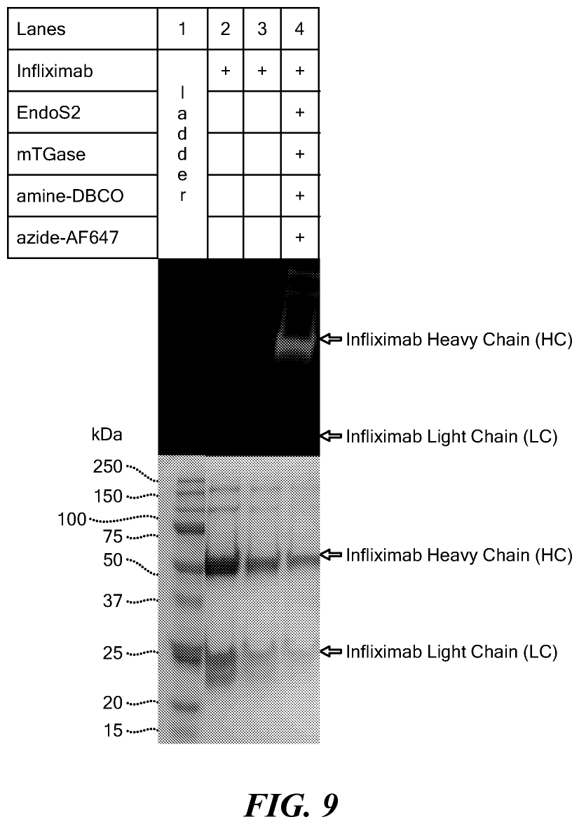

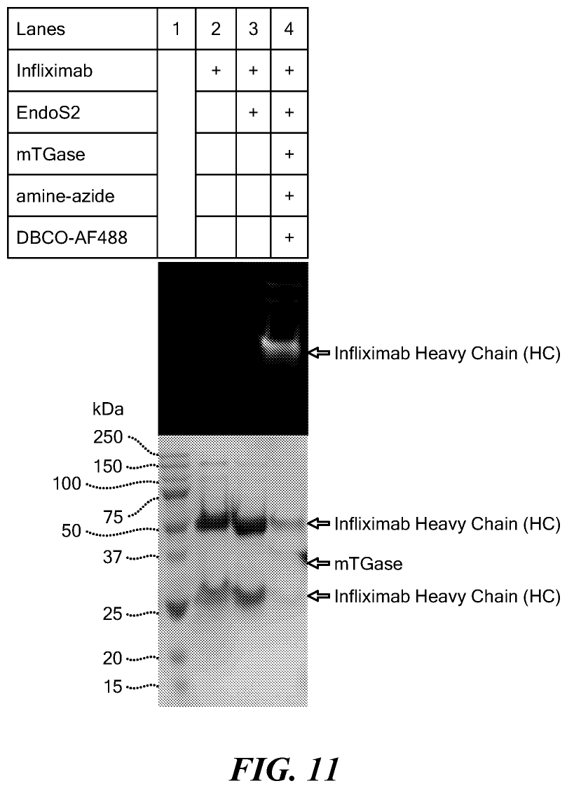

Infliximab (Remicade) was tested and showed similar results (FIG. 9, FIG. 11), as expected from the near identical structures in the Fc regions for these antibodies.

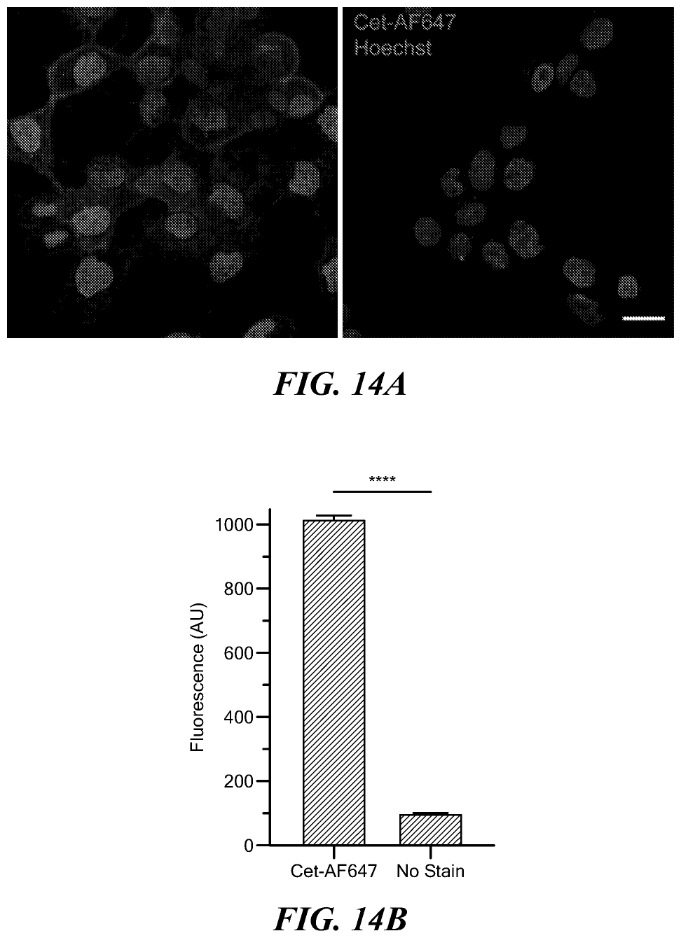

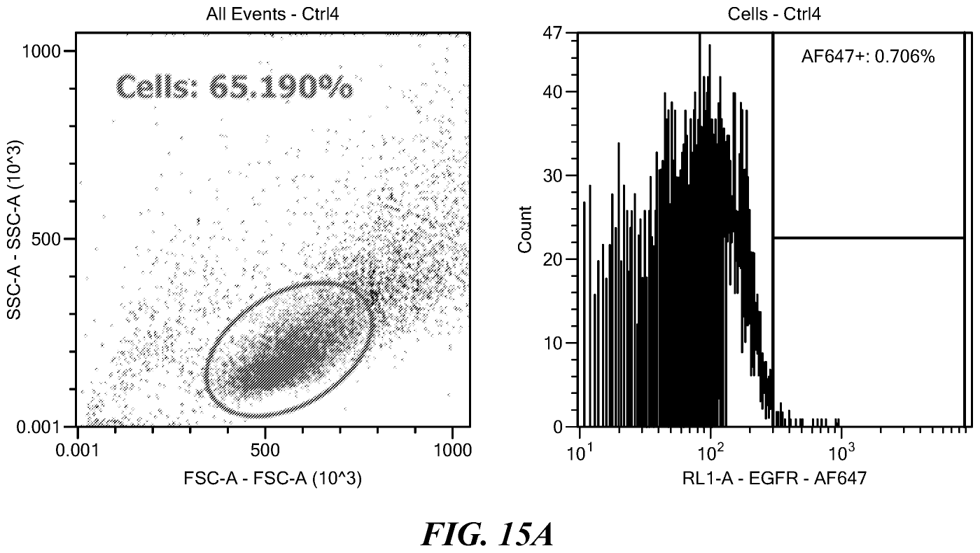

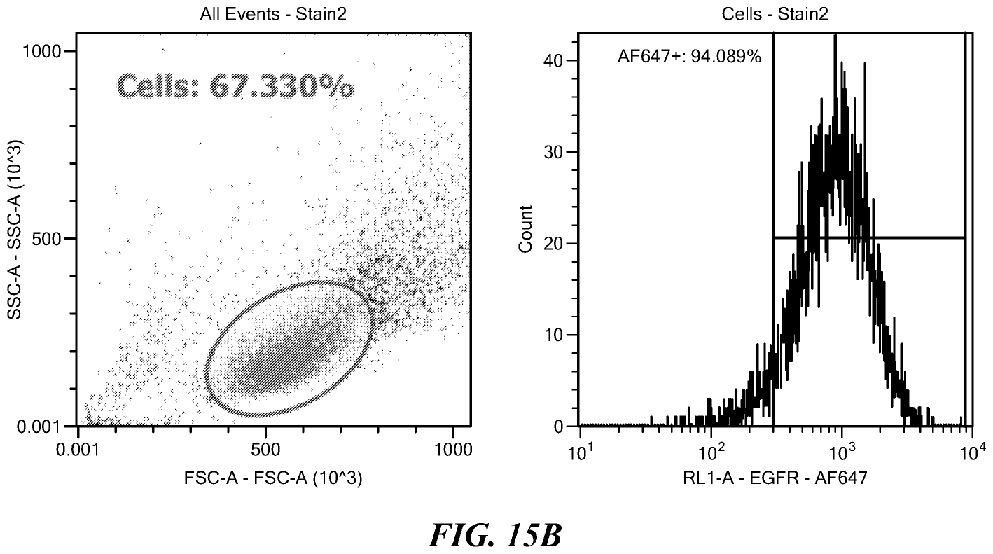

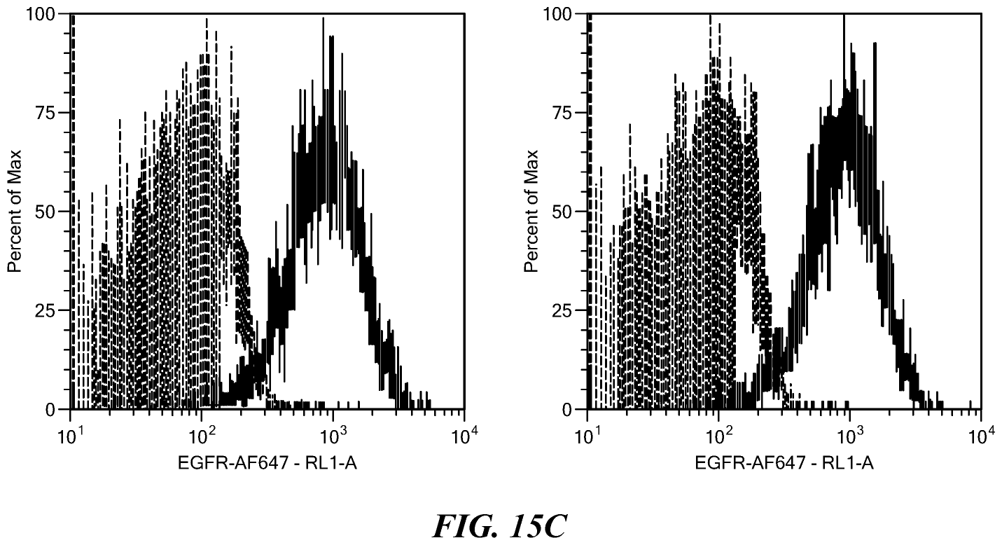

The biological activity of antibody conjugates, i.e., cetuximab conjugated to Alexa Fluor647 (FIG. 14A), is assessed via confocal fluorescence microscopy in a cancer cell line (Ovcar3) that highly expresses epidermal growth factor receptor (EGFR). Under physiological conditions, the antibody conjugates bind and internalize into the cytoplasm of Ovcar3, as evident from the fluorescence signal (FIG. 14A-B, FIG. 15A-C). These data confirm that the conjugate's biological activity is preserved.

Non-specific methods, which include acylation of amines (e.g., lysines and the N-terminus) or alkylation of thiols (e.g., cysteines), have been used to assemble various glycoproteins. Some glycoproteins are approved by the U.S. Food and Drug Administration such as antibody drug conjugates (ADCs) (Chari, et al., 2014; Beck, et al., 2017). Over 100 ADCs are currently in clinical trials, but specific methods of making ADCs are urgently needed.

The previous methods for modifying glycoproteins have several limitations. First, the reactions lack site-specificity and result in heterogenous mixtures. In general, kinetically controlled, these processes also suffer from poor reproducibility and are prone to side-reactions (Luo, et al., 2016). Second, modification at or near the functional domains such as the complementarity-determining regions of antibodies is likely to perturb the structure and thus function. These multiple sites of modification have been shown to affect the protein's activity (e.g., antibody) negatively, for example, reducing the binding affinity and specificity. Third and lastly, characterization of non-specific conjugation is cumbersome, as before conjugation, many proteins already have various post-translation modifications (PTMs) such as deamidation, oxidation, glycosylation, cross-linking, or other reactive metabolites. See, e.g., Liu et al., 2016; Chumsae et al., 2015; Chumsae et al., 2014; Klaene et al., 2014; Liu et al., 2014; Chumsae et al., 2013; and Dai et al., 2013. Accounting for these PTMs, the number of unmodified and modified species increases exponentially after non-specific bioconjugation. This synthetic approach makes it difficult to detect all modifications and often leads to underestimation. For example, sites of modification are missed because of the prevalence of false negatives during analysis. To overcome these limitations, the technology disclosed herein provides site-specific methods that are predictable, can be carried out with few steps and offer new possibilities for glycoprotein engineering.

### EXAMPLES

### Example 1. Trimming of N-glycan Using Endoglycosidase (EndoS)

The hydrolysis of N-glycans using cetuximab (Erbitux, chimeric IgG1) was examined as a model system. Both endoglycosidase (EndoS) and amidase (PNGase F) hydrolyzed N-glycans on the constant domain (Fc) of cetuximab and various antibodies (IgG1-4), albeit with several differences. First, hydrolysis or trimming by EndoS (an endoglycosidase; EndoS and EndoS2 are used interchangeably) retained the innermost glycan N-acetylglucosamine (GlcNAc), whereas hydrolysis by PNGase F removed the glycan completely. Second, hydrolysis by EndoS retained the antibody's asparagine residue, whereas hydrolysis by PNGase F (an amidase) underwent deamidation which led to conversion of asparagine residue into aspartic acid (Asp297, carboxylic acid sidechain, FIG. 1A, top).

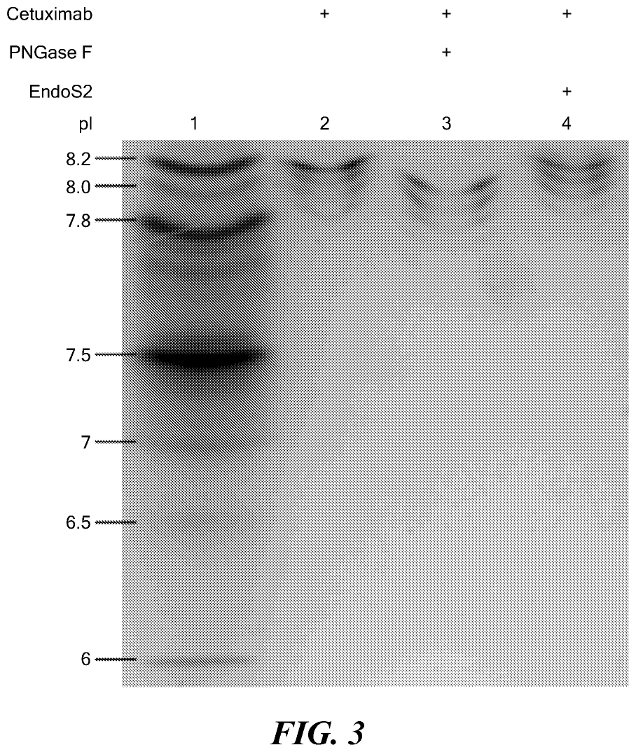

A comparison of PNGase F and EndoS mediated hydrolysis of cetuximab is presented in FIG. 3. As demonstrated in FIG. 3, these processes were confirmed using an isoelectric focusing (IEF) gel. Isoelectric focusing gel illustrated a shift in the overall pI of the antibody (i.e., charge profile) after PNGase F hydrolysis (lane 3, FIG. 3), whereas EndoS hydrolysis (lane 4) of the antibody maintained an indistinguishable pI or charge variant profile to their native counterpart (lane 2).

The isoelectric points (pI) of the EndoS-treated antibody (pI 7.8-8.2; FIG. 3, lane 4) were indistinguishable to the unmodified antibody (pI 7.8-8.2; FIG. 3, lane 2) and lower (more acidic) for the PNGase F treated antibody (pI 7.6-8.0; FIG. 3, lane 3). The pI difference is consistent with the deamidation mediated by PNGase F, resulting in a ˜0.2 difference shift in the pI. The multiple bands for the unmodified antibody (pI 7.8 - 8.2; FIG. 3, lane 2) are a result of intrinsic charge heterogeneity. These variants were a result of various post-translational modifications (PTMs) such as deamidation, C-terminal lysine, glycosylation or other reactive metabolites. Based on the site-specificity, it was concluded that many antibodies (e.g., human IgG 1, 2, 3 and 4) are amenable to hydrolysis or trimming of the N-glycan catalyzed by EndoS (Allhorn, M., et al., 2008).

Subsequent site-specific TGase-mediated conjugation of polyethylene glycol (PEG) and chromophore (fluorophore) onto cetuximab is shown in FIG. 5A. EndoS mediated reactions had near quantitative conversions, as observed by clean downward shifts in the electrophoretic mobility of the heavy chain (HC) of the antibody using SDS-PAGE (FIG. 5A, lane 2). In FIG. 5A, SDS-PAGE analysis showed near quantitative installing of a 10 kDa PEG groups onto antibody (lanes 3 and 4).

Additional trimming reactions were carried out to study transamidation. The reactions contained 18 μM cetuximab (Erbitux, Selleckchem, A2000) or 17 μM Infliximab (Remicade, European Pharmacopoeia, Y0002047) and 25 mM tris-buffered saline (TBS) pH 7.4, and was initiated with immobilized EndoS2 GlycINATOR (E.C. 3.2.1.96, Genovis; AO-GL6-010, per manufacturer instructions) at 37° C., and for 3 h and 40 min. To remove excess unreacted reagents, each reaction mixture was desalted using 30 kDa molecular weight cut-off (MWCO) centrifugal filters (Amicon unit, UFC503096) into 25 mM TBS pH 7.4, prior to the transamidation reaction.

### Example 2. Transglutaminase-Catalyzed Conjugation of EndoS-Treated Antibody

After Endo-S mediated trimming of glycan in cetuximab, an evaluation of various amine-containing substrates using microbial transglutaminase (mTGase), which converts unsubstituted amides on glutamines into substituted amides, was conducted. This enzyme was utilized because it has been shown that mTGase has broad specificity towards the amines with two main features: primary amines are generally accepted and substituents at the alpha position significantly slows the reaction down (Gundersen, M., et al., 2014). All amine-containing molecules evaluated were excellent substrates for mTGase (e.g., compounds 1 and 2; FIG. 4B).

The transamidation reaction contained 25 mM TBS pH 7.4, 100 mM 2-azidoethanamine (compound 1, FIG. 4B; Acrotein ChemBio, AS00696) or 5 mM dibenzylcyclooctyne-PEG4-amine (compound 2, FIG. 4B; Click Chemistry Tools, A103P), and 1.6 μM trimmed cetuximab or 1.5 μM trimmed Infliximab, and was initiated with 1.7 μM microbial transglutaminase (mTGase, EC 2.3.2.13, Uniprot P81453; Ajinomoto, ACTIVA-TI formulation) and incubated at 37° C. for 12.5 h. To remove excess unreacted reagents, each reaction mixture was desalted using 50 kDa MWCO centrifugal filters (Amicon unit, UFC505024) into 25 mM TBS pH 7.4, prior to a click reaction.

The concentrations of the peptides and proteins were determined using UV absorption at 280 nm and extinction coefficients based on amino acid sequences. All aqueous solutions were prepared using Milli-Q water. All cell culture methods were performed with aseptic technique in a biosafety cabinet.

As illustrated in FIG. 4A, antibodies conjugated with an amine-containing clickable handle were further derivatized via click chemistry. Examples of clickable handles are shown in FIG. 4B for assembly of the antibody conjugates via strain-promoted azide-alkyne cycloaddition (SPAAC) click chemistry. The trimmed antibody is conjugated to install an amine-containing azide (compound 1, FIG. 4B) or cyclooctyne (compound 2)—into unsubstituted amide of glutamine (Gln) TGase. Modified antibody intermediate is further derivatized to a chromophore with a complementary clickable handle—AF488 (compound 3), AF647 (compound 4) or DBCO-PEG-10 kDa (compound 5). The cyclooctyne and azide form a triazole linker (compounds 6, 7 and 8) in the final conjugates.

These modifications were confirmed by attachment of 10 kDa polyethylene glycol (PEG-10 kDa, compound 4, FIG. 4B) and various fluorophores (compounds 3 and 5, FIG. 4B) onto cetuximab via a two-step click chemistry reaction (FIG. 4B), namely SPAAC (Agard, N., et al., 2004). Similarly, other click reactions can be utilized in a similar fashion such as tetrazine ligation or copper-catalyzed azide-alkyne cycloaddition (CuAAC) (Rostovtsev, V. V., et al., 2002).

The SPAAC reaction contained 25 mM TBS pH 7.4, 200 μM Alexa Fluor 647® azide (AF647, compound 3, FIG. 4B; Click Chemistry Tools, 1299) or Alexa Fluor 488® dibenzylcyclooctyne (AF488, compound 4, FIG. 4B; Genovis, L1-F01-025, per manufacturer instructions) or 10 kDa polyethylene glycol (PEG) dibenzylcyclooctyne (DBCO-PEG-10 kDa, compound 5, FIG. 4B; Click Chemistry Tools, A119), and 1.5 μM cetuximab or Infliximab modified by mTGase. To remove excess unreacted reagents, each reaction mixture was desalted using 50 kDa MWCO centrifugal filters into 25 mM TBS pH 7.4.

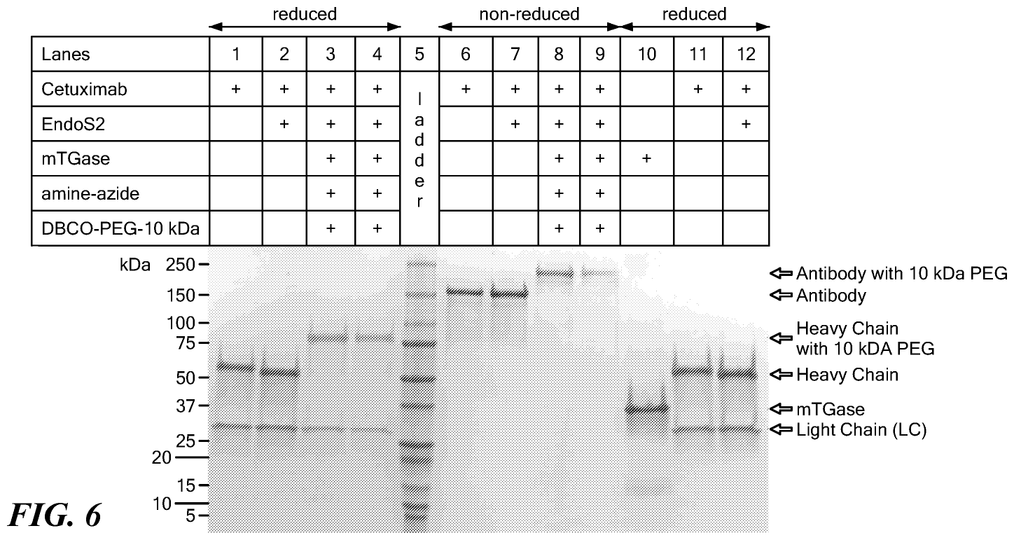

The reaction was quantitative, as determined by a complete shift in the electrophoretic mobility of the antibody's heavy chain (HC) conjugated to a 10 kDa PEG (FIG. 5A, FIG. 6). The unmodified heavy chain (HC) of the antibody was not observed in FIG. 5A and FIG. 6.

FIG. 5A, and FIG. 5B show the site-specific TGase-mediated conjugation of polyethylene glycol (PEG) and chromophore (fluorophore) onto cetuximab. The SDS-PAGE analysis in FIG. 5A showed near quantitative installing of a 10 kDa PEG groups onto antibody (lanes 3 and 4). The SDS-PAGE analysis of antibody-fluorophore conjugates in FIG. 5B showed modification primarily at the heavy chain (HC) of the antibody, i.e., no modification of the light chain (LC) was detected.

FIG. 6 shows SDS-PAGE of the cetuximab-PEGylated conjugates with Coomassie staining. The lanes are as follows: reduced, 1) cetuximab, 2) cetuximab treated with endoglycosidase (EndoS2), 3) and 4) trimmed cetuximab incubated with transglutaminase (mTGase), 2-azidoethanamine, and then 10 kDa polyethylene glycol (PEG) dibenzylcyclooctyne (DBCO-PEG-10 kDa), 5) molecular weight standards; non-reduced, 6) cetuximab, 7) cetuximab treated with Endoglycosidase (EndoS2), 8) trimmed cetuximab incubated with transglutaminase (mTGase), 2-azidoethanamine, and then 10 kDa polyethylene glycol (PEG) dibenzylcyclooctyne (DBCO-PEG-10 kDa); reduced, 9) cetuximab, 10) cetuximab treated with Endoglycosidase (EndoS2). Based on the shift by PEG (lanes 3, 4, 8 and 9), the TGase-mediated and click chemistry reactions yields were quantitative.

Cetuximab contains complex biantennary Fc N-glycans that are highly heterogenous (both fucosylated and non-fucosylated exist) and most species are fucosylated (Qian, J., et al., 2007). Since the overall yield was quantitative, it was deduced that both fucosylated and non-fucosylated or afucosylated glycan species were modified, which further expanded the utility of the approach.

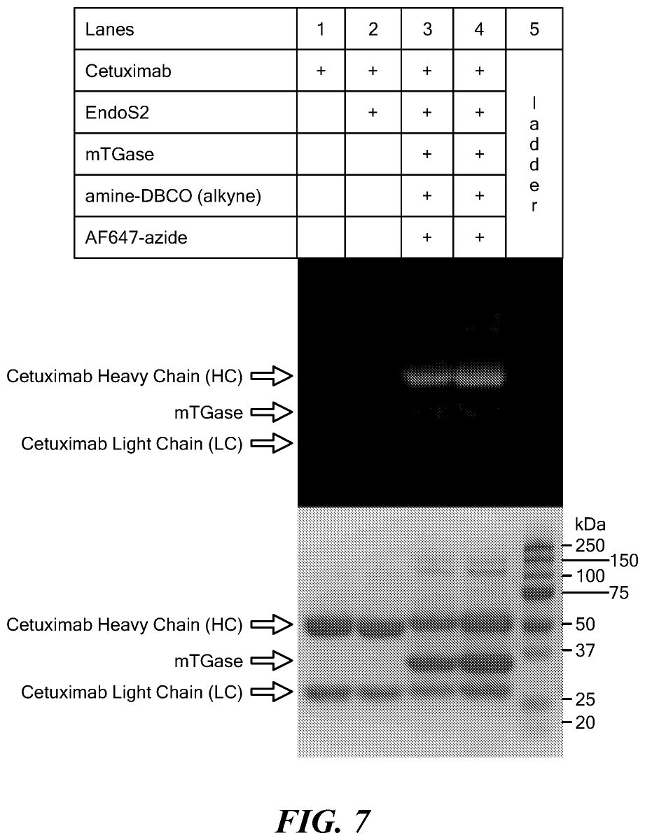

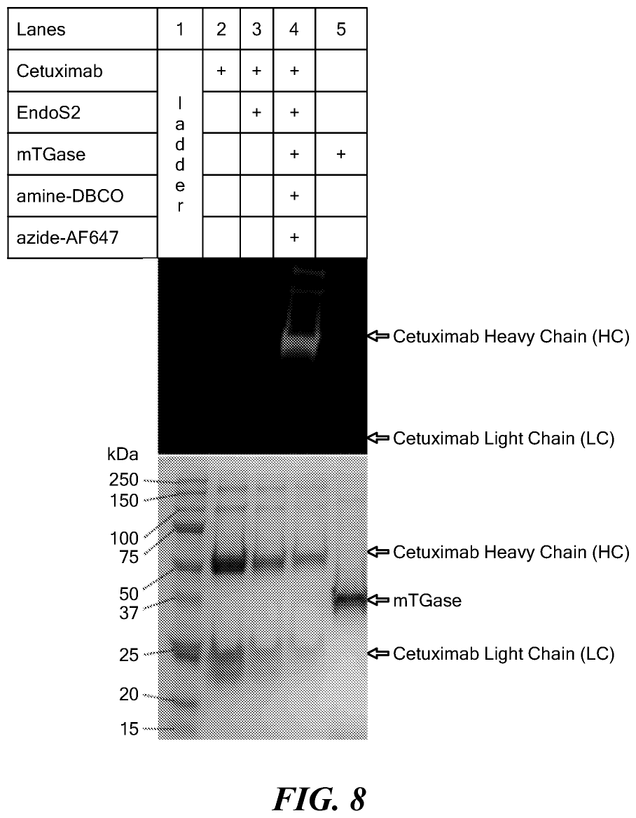

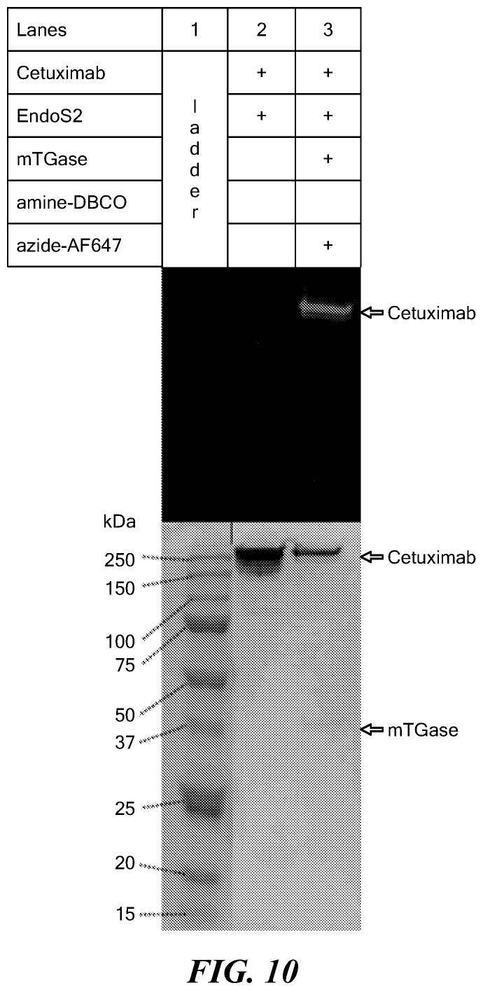

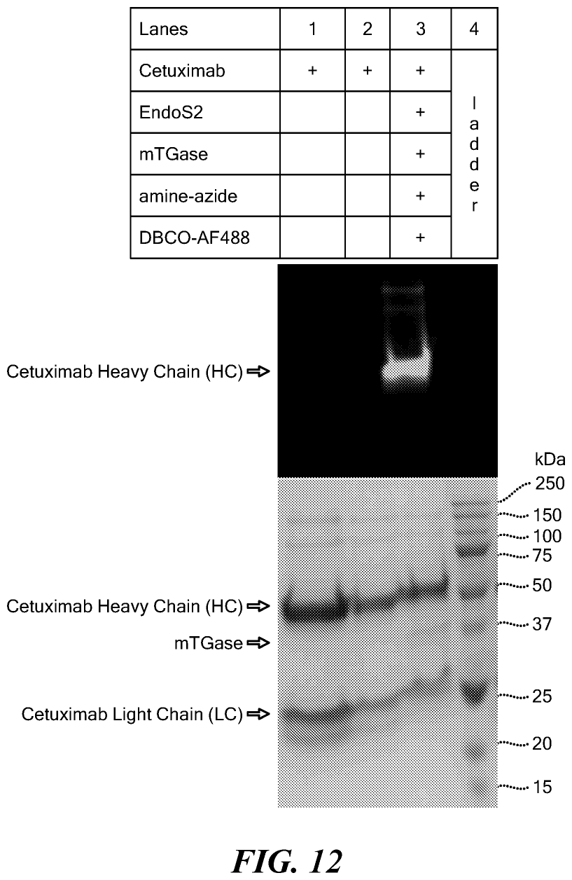

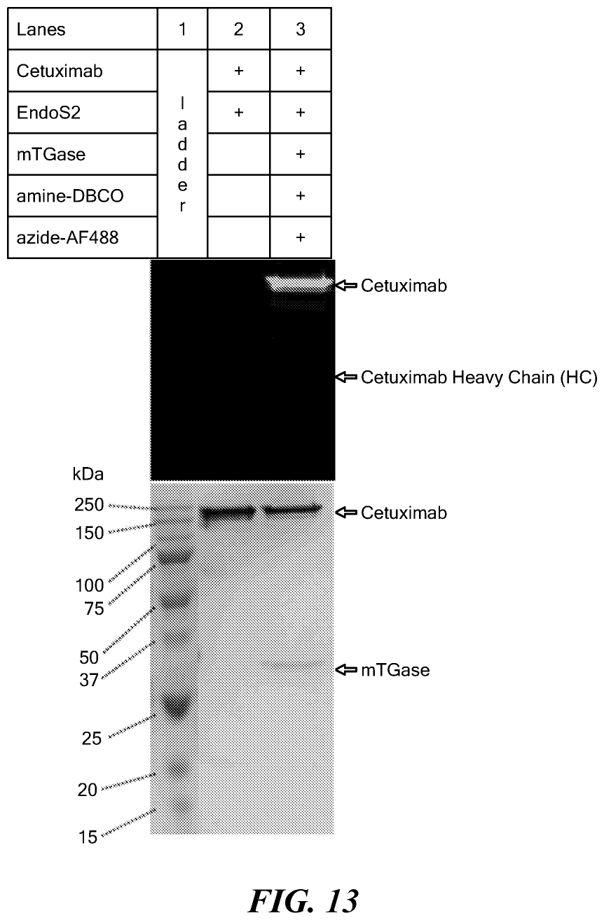

The TGase-catalyzed transformation resulted in a modification at a single site [glutamine (Gln, Q) 295] in the heavy chain (HC) of the antibody (FIG. 5B, FIG. 7, FIG. 8, FIG. 10, FIG. 12 and FIG. 13). Under the reaction conditions, the load (i.e., chromophore) ratio was approximately one (Table 2), as determined by ultra-violet (UV) spectroscopy.

TABLE 2
Antibody conjugates' chromophores loading ratio.

| | Target/Antibody | | | |
|---|---|---|---|---|
| | EGFR/Cetuximab | | TNF-α/Infliximab | |
| Chromophore | AlexaFluor 647 | AlexaFluor 488 | AlexaFluor 647 | Alexa Fluor 488 |
| (Ex/Em (nm)) | (650/668) | (495/519) | (650/668) | (495/519) |
| Antibody- | 0.70 ± 0.05 | 0.61 ± 0.02 | 0.74 ± 0.00 | 0.79 ± 0.05 |
| chromophore | | | | |
| ratio | | | | |

The loading ratio was calculated based on the ratio of the absorption of the chromophore and antibody. The absorbance spectra of antibody-chromophore conjugates were collected from 220 to 750 nm (NanoDrop spectrophotometer, ND-1000). The concentration of the chromophore AF647 or AF488 was calculated using the absorbance of the conjugates at 650 nm or 495 nm and extinction coefficient of 270,000 M⁻¹cm⁻¹ or 73,000 M⁻¹cm⁻¹ respectively, provided by Click Chemistry Tools. The antibody concentration was estimated by subtracting the chromophores contribution at 280 nm and the calculated extinction coefficient based on its amino acid sequence (217,440 M⁻¹cm⁻¹ for cetuximab; 203585 M⁻¹cm⁻¹ for Infliximab). The summary of each conjugate's ratio is provided in Table 2.

Reducing SDS-PAGE of the cetuximab-chromophore conjugates is shown in FIG. 7. At the top of FIG. 7, fluorescence imaging was with excitation at 608-632 nm and emission at 675-720 nm. At the bottom of FIG. 7 is the Coomassie staining. The lanes are as follows: 1) cetuximab, 2) cetuximab treated with Endoglycosidase (EndoS2), 3) and 4) trimmed cetuximab incubated with transglutaminase (mTGase) and dibenzylcyclooctyne-PEG4-amine, and then Alexa Fluor 647® azide, 5) molecular weight standards.

Reducing SDS-PAGE of the cetuximab-chromophore conjugates is shown in FIG. 8. At the top of FIG. 8 is the fluorescence imaging (excitation 608-632 nm and emission 675-720 nm). At the bottom of FIG. 8 is the Coomassie staining. Lanes: 1) molecular weight standards, 2) cetuximab, 3) cetuximab treated with endoglycosidase (EndoS2), 4) trimmed cetuximab incubated with transglutaminase (mTGase) and dibenzylcyclooctyne-PEG4-amine, and then Alexa Fluor 647® azide, 5) mTGase.

Nonreducing SDS-PAGE of the cetuximab-chromophore conjugates is shown in FIG. 10. At the top of FIG. 10 is the fluorescence imaging (excitation 608-632 nm and emission 675-720 nm). The Coomassie staining is at the bottom of FIG. 10. Lanes: 1) cetuximab treated with Endoglycosidase (EndoS2), 2) trimmed cetuximab incubated with TGase and dibenzylcyclooctyne-PEG4-amine, and then Alexa Fluor 647 azide.

Reducing SDS-PAGE of the cetuximab-chromophore conjugates is shown in FIG. 12. At the top is the fluorescence imaging (excitation 455-485 nm and emission 508-557 nm). At the bottom is the Coomassie staining. Lanes: 1) cetuximab, 2) cetuximab treated with Endoglycosidase (EndoS2), 3) trimmed cetuximab incubated with TGase and 2-azidoethanamine, and then Alexa Fluor 488 dibenzylcyclooctyne, 4) Molecular Weight standards.

Nonreducing SDS-PAGE of the cetuximab-chromophore conjugates is shown in FIG. 13: (a) top, fluorescence imaging (excitation 455-485 nm and emission 508-557 nm), and (b) bottom, Coomassie staining. Lanes: 1) cetuximab treated with EndoS2, 2) trimmed cetuximab incubated with TGase and 2-azidoethanamine, and then Alexa Fluor 488 dibenzylcyclooctyne.

The enzymatic reaction can be further optimized to increase the DAR by increasing the temperature, reaction time or enzyme concentration to achieve a maximum loading ratio of 2, as described in the literature (Jeger, S., et al., 2010; Dennler, P., et al., 2014). To further expand the scope, Infliximab (or Remicade) was evaluated and found that various amine-containing clickable handles and fluorophores (FIG. 9 and FIG. 11), were incorporated in a two-step SPAAC reaction. Similar results were obtained.

Sodium dodecyl sulfate-polyacrylamide gel electrophoresis (SDS-PAGE) was performed using a Bio-Rad Mini-PROTEAN 3 system or Criterion Cell. First, the reaction mixture was incubated with SDS Sample Buffer at 80° C. for 10 min. For reducing and nonreducing gels, 4× reducing SDS sample buffer (Boston Bio Products, BP-110R) and 2× nonreducing SDS sample buffer (Bio-Rad, 1610737) were used, respectively. Second, the samples were loaded into 12% tris-tricine precast protein gels (Bio-Rad, 4561044) or 4-15% tris-glycine precast protein gels (Biorad, 5671083). Precision Plus Protein™ Dual Xtra Prestained Protein Standards (Bio-Rad, 1610377) were used for mass calibration. Electrophoresis was then performed at 200 V for 20 min. The gel was stained by Coomassie R250 and then destained using 10% acetic acid and 40% methanol. The gels were imaged using an iBright FL1000 Imaging system (Thermo Fisher Scientific).

Reducing SDS-PAGE of infliximab-chromophore conjugates is shown in FIG. 9: (a) top, fluorescence imaging (excitation 608-632 nm and emission 675-720 nm), and (b) bottom, Coomassie staining. Lanes: 1) Molecular Weight standards, 2) Infliximab, 3) Infliximab treated with Endoglycosidase (EndoS2), 4) trimmed Infliximab incubated with transglutaminase (mTGase) and dibenzylcyclooctyne-PEG4-amine, and then Alexa Fluor 647 azide.

Reducing SDS-PAGE of infliximab-chromophore conjugates is shown in FIG. 11: (a) top, fluorescence imaging (excitation 455-485 nm and emission 508-557 nm), and (b) bottom, Coomassie staining. Lanes: 1) Molecular Weight standards, 2) Infliximab, 3) Infliximab treated with Endoglycosidase (EndoS2), 4) trimmed Infliximab incubated with transglutaminase (TGase) and 2-azidoethanamine, and then Alexa Fluor 488 dibenzylcyclooctyne.

Isoelectric focusing was performed using a Bio-Rad Criterion system. First, the reaction mixture was mixed in 1:1 dilution with isoelectric focusing sample buffer (Bio-Rad, 1610763). Second, the samples were loaded into pH 3-10 Criterion isoelectric focusing precast gel and placed into the cell. Then, 1× Anode buffer (Bio-Rad, 1610761) and 1× Cathode buffer (Bio-Rad, 1610762) were placed in the upper and lower chamber of the criterion cell, respectively. Third, isoelectric focusing was run initially at 100 V for 1 h to initiate desalting of the sample, followed by higher voltage at 250 V for 1 h to mobilize the antibody, and lastly 500V for 30 min to complete electro-focusing. The gel was stained by Coomassie R250 and Crocein Scarlet and then destained using 10% acetic acid and 40% methanol. The gels were imaged using an iBright FL1000 Imaging system.

### Example 3. Cell-Based Activity in an Ovarian Cancer Model

NIH:OVCAR-3 (Ovcar3) ovarian carcinoma cells (HTB-161, American Type Culture Collection, ATCC) were cultured in the recommended RPMI-1640 media (ATCC, 30-2001) supplemented with 20% heat-inactivated fetal bovine serum (R&D Systems, S11150H) and 0.01 mg/mL bovine insulin (Sigma-Aldrich, I0516) in a humidified incubator at 37° C. and 5% CO₂. Media was replenished every 2-3 days and cells were passaged in T75 culture-treated flasks (Thermo Scientific, 12-565-350) at 70-90% confluency.

Ovcar3 cells were harvested and plated in a 24-well plate with #1.5 cover glass (P24-1.5H-N, Cellvis) at 30,000 cells per well in 1 mL of cell culture media. Cells were incubated for 48 h. Cet-AF647 staining solution was prepared by diluting Cet-AF647 to 5 ng/μL in cell culture media. Media from each well was aspirated and replaced with 200 μL of 5.0 ng/μL staining solution or with fresh media and incubated for 1 h at 37° C. prior to imaging. 15 min prior to imaging, 2.5 μg/mL of Hoechst 33342 (Invitrogen, H3570) was added to each well. The plate was then imaged using confocal fluorescence microscopy (Zeiss LSM800) with a 40× objective. All imaging parameters were kept consistent throughout imaging.

The biological activity of the antibody conjugates, i.e., cetuximab conjugated to Alexa Fluor 647, was assessed via confocal fluorescence microscopy in a cancer cell line (Ovcar3) that highly expresses epidermal growth factor receptor (EGFR). Under physiological conditions, the antibody conjugates bound and internalized into the cytoplasm of Ovcar3, as evident from the fluorescence signal (FIG. 14A and FIG. 14B).

This data confirmed that the conjugate's biological activity was preserved. FIG. 14A shows ovarian cancer cells (Ovcar3) were stained with cetuximab fluorophores conjugates (Cet-AF647, FIG. 14A, left) or without (FIG. 14A, right) and imaged via confocal microscopy. Both groups were stained with Hoechst 33342 nuclear stain. Fluorescence is apparent in the plasma membrane and cytoplasm, indicating that Cet-AF647 bound and internalized. The scale bar at the right of FIG. 14A is 20 μm. The Ovcar3 cells stained with Cet-AF647 and analyzed with flow cytometry are 10-fold brighter than unstained cells as demonstrated in FIG. 14B (Unpaired, two-tailed t-test, ****P<0.0001). Results are mean(±S.D.) fluorescence intensity (n=6 replicates). This data confirmed that the conjugate's biological activity was preserved. Further data was collected and summarized in FIG. 15A, FIG. 15B, and FIG. 15C described below.

### Example 4. Antibody Conjugates Activity via Flow Cytometry

Ovcar3 cells were harvested and resuspended at 5×10⁵ cells/mL in 1 mL of media with and without 0.5 ng/μL, of cetuximab-AF647 (Cet-AF647). Cells were incubated for 1 hour at 4° C. in the dark. Each sample was washed twice and resuspended in 1 mL of phosphate buffered saline (10-010-023, Gibco). Cells were analyzed via flow cytometer (Attune NxT, Thermo Fisher) equipped with 635 nm laser. Stained and unstained cells were sampled 6 times. Cells were gated and measured for mean AF647 fluorescence using Attune NxT software. Results were compiled and analyzed in Prism 8 (GraphPad Software).

FIG. 15A, FIG. 15B, and FIG. 15C shows analysis of the Ovcar3 cells via flow cytometry. FIG. 15A and FIG. 15B show exemplary forward scatter (FSC) and side scatter (SSC) dot plots of raw data (top) showing cell gating scheme and AF647 fluorescence histograms (bottom) for unstained cells (FIG. 15A) and cells stained with Cet-AF647 (FIG. 15B). Two exemplary overlays comparing stained and unstained cell populations are shown in FIG. 15C. Each group was sampled 6 times and mean fluorescence intensity was recorded for further analysis. An example of further analysis is shown in FIG. 14B.

### Example 5. Characterization of the Conjugates by Thermal Shift Assay

All thermal stability assays were carried out using a 96-well plate with a final volume of 20 μL on a Bio-Rad CFX96 Real-Time PCR Detection System. Samples for all antibody conjugates were prepared at concentrations of 0.2 mg/mL. The assays were performed in PBS pH 7.4 with a 10× concentration of SYPRO Orange (Invitrogen). Samples were heated from 20 to 100° C. at a rate of 0.5° C./min with fluorescence measurements recorded at 0.2-degree increments. Melting temperatures were calculated from ΔF/ΔT values based on a minimum of three replicates. Standard deviation is reported with listed values in Table 3 below.

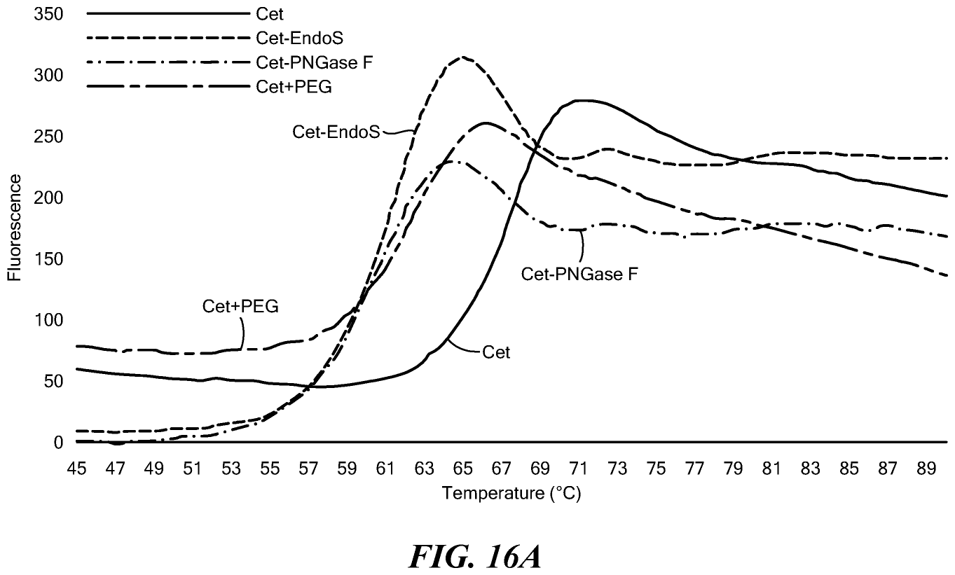

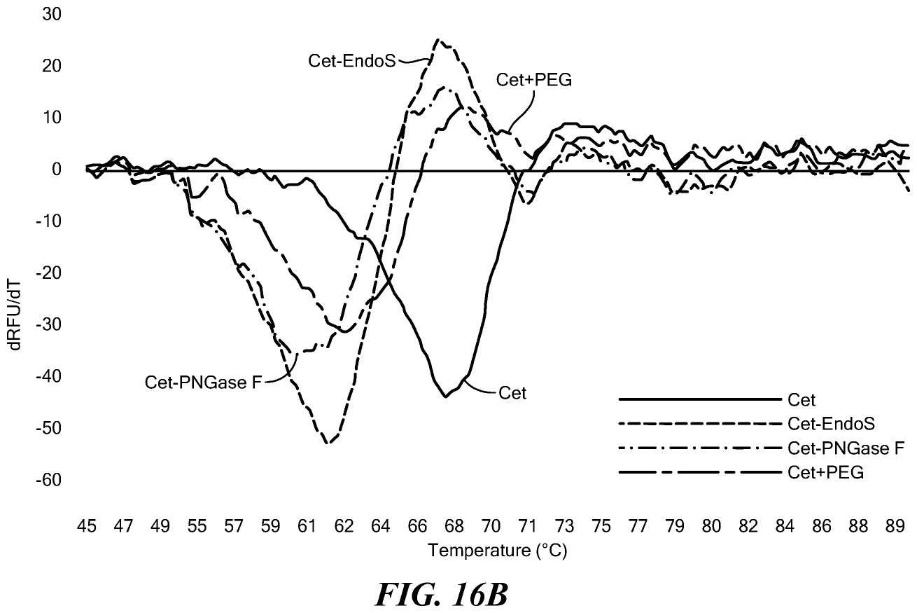

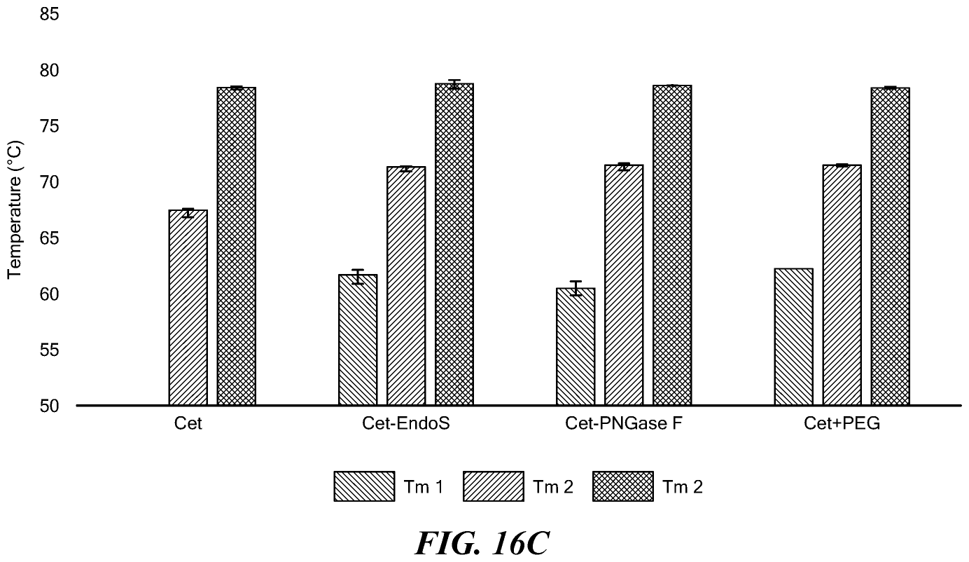

FIGS. 16A and FIG. 16B show thermal shift stability assay data of cetuximab (Cet); cetuximab treated with EndoS2 (Cet-EndoS2, which was also named trimmed cetuximab); cetuximab treated with PNGase F (Cet-PNGase F) and trimmed cetuximab incubated with mTGase, 2-azidoethanamine, and then 10 kDa DBCO-PEG-10 kDa (Cet-PEG). FIG. 16C shows a summary plot of melting temperatures (Tm) obtained from the assay. Table 3 shows the summary of melting temperatures (Tm) obtained from the assay. Melting temperatures were assessed using Sypro Orange dye fluorescence.

TABLE 3
Summary of Melting Temperatures

| | Tm 1 (° C.) | Tm 2 (° C.) | Tm 3 (° C.) |
|---|---|---|---|
| Cet | Not detected | 67.37 ± 0.31 | 78.50 ± 0.15 |
| Cet-EndoS2 | 61.60 ± 0.16 | 71.27 ± 0.19 | 78.87 ± 0.38 |
| Cet-PNGase F | 60.60 ± 0.57 | 71.47 ± 0.34 | 78.60 ± 0.00 |
| Cet-PEG | 62.33 ± 0.09 | 71.67 ± 0.09 | 78.53 ± 0.09 |

U.S. Pat. No. 11,129,790B2 is hereby incorporated by reference in its entirety.

### References

Anami, Y., Xiong, W., Gui, X., Deng, M., Zhang, C. C., Zhang, N., An, Z., and Tsuchikama, K. (2017) Enzymatic conjugation using branched linkers for constructing homogeneous antibody-drug conjugates with high potency. Org Biomol Chem 15, 5635-5642. Chari, R. V., Miller, M. L., and Widdison, W. C. (2014) Antibody-drug conjugates: an emerging concept in cancer therapy. Angew Chem Int Ed Engl 53, 3796-827.
Beck, A., Goetsch, L., Dumontet, C., and Corvaia, N. (2017) Strategies and challenges for the next generation of antibody-drug conjugates. Nat Rev Drug Discov 16, 315-337.
Chumsae C., Gifford K., Lian W, Liu H., Radziejewski C. H., Zhou Z. S. Arginine Modifications by Methylglyoxal: Discovery in a Recombinant Monoclonal Antibody and Contribution to Acidic Species. Analytical Chemistry, 2013, 85, 11401-9.
Chumsae, C., Hossler P, Raharimampionona H., Zhou Y., McDermott S., Racicot C., Radziejewski C., Zhou, Z. S. When Good Intentions Go Awry: Modification of a Recombinant Monoclonal Antibody in Chemically Defined Cell Culture by Xylosone, an Oxidative Product of Ascorbic Acid. Analytical Chemistry, 2015, 87, 7529-7534.
Chumsae C., Zhou L. L., Shen Y., Wohlgemuth J., Fung E., Burton R., Radziejewski C., Zhou Z. S. Discovery of a Chemical Modification by Citric Acid in a Recombinant Monoclonal Antibody. Analytical Chemistry, 2014, 86, 8932-8936.
Collin, M., and Olsen, A. (2001) EndoS, a novel secreted protein from Streptococcus pyogenes with endoglycosidase activity on human IgG. EMBO J 20, 3046-55.
Cunningham O., Scott M., Zhou Z. S. and Finlay W. J. J. Polyreactivity and Polyspecificity in Therapeutic Antibody Development: Significant Risks for Failure in Preclinical and Clinical Development Campaigns. mAbs, 2021, 13, 13, 1999195.
Dennler, P., Chiotellis, A., Fischer, E., Bregeon, D., Belmant, C., Gauthier, L., Lhospice, F., Romagne, F., and Schibli, R. (2014) Transglutaminase-based chemo-enzymatic conjugation approach yields homogeneous antibody-drug conjugates. Bioconjug Chem 25, 569-78.
EMBL-EBI Clustal Omega (ebi.ac.uk/Tools/msa/clustalo/).
Fujita K, Kiyotaka Fujita, Fusako Oura, Noriko Nagamine, Takane Katayama, Jun Hiratake, Kanzo Sakata, Hidehiko Kumagai, Kenji Yamamoto, Identification and Molecular Cloning of a Novel Glycoside Hydrolase Family of Core 1 Type O-Glycan-Specific Endo-α-N-acetylgalactosaminidase from Bifidobacterium longum* Glycobiology And Extracellular Matrices, Vol. 280, 45, P37415-37422, November 2005.
Gao, P., Pinkston, K. L., Wilganowski, N., Robinson, H., Azhdarinia, A., Zhu, B., Sevick-Muraca, E. M., and Harvey, B. R. (2015) Deglycosylation of mAb by EndoS for improved molecular imaging. Mol Imaging Biol 17, 195-203.
Jeger, S., Zimmermann, K., Blanc, A., Grunberg, J., Honer, M., Hunziker, P., Struthers, H., and Schibli, R. (2010) Site-specific and stoichiometric modification of antibodies by bacterial transglutaminase. Angew Chem Int Ed Engl 49, 9995-7.
Josten, A., Haalck, L., Spener, F., and Meusel, M. (2000) Use of microbial transglutaminase for the enzymatic biotinylation of antibodies. J Immunol Methods 240, 47-54.
Klaene J. J., Ni W., Alfaro J. F., Zhou Z. S. Detection and Quantitation of Succinimide in Intact Protein via Hydrazine Trapping and Chemical Derivatization. Journal of Pharmaceutical Science, 2014, 103, 3033-3042.
Koutsioulis D, Landry D, Guthrie E P. Novel endo-alpha-N-acetylgalactosaminidases with broader substrate specificity. Glycobiology. 2008 October; 18(10):799-805. doi: 10.1093/glycob/cwn069. Epub 2008 Jul. 17. PMID: 18635885; PMCID: PMC2553423.
Liu M., Zhang Z., Cheetham J., Ren D., Zhou Z. S. Discovery and Characterization of a Novel Photo-Oxidative Histidine-Histidine Crosslink in IgG1 Antibody Utilizing 18O-labeling and Mass Spectrometry. Analytical Chemistry, 2014, 86, 4940-8
Liu S., Moulton K. R., Auclair J. R., Zhou, Z. S. Mildly Acidic Conditions Eliminate Deamidation Artifact during Proteolysis: Digestion with Endoprotease Glu-C at pH 4.5. Amino Acids, 2016, 48, 1059-67.
Luo, Q., Chung, H. H., Borths, C., Janson, M., Wen, J., Joubert, M. K., and Wypych, J. (2016) Structural Characterization of a Monoclonal Antibody-Maytansinoid Immunoconjugate. Anal Chem 88, 695-702.
Manabe, S., Yamaguchi, Y., Matsumoto, K., Fuchigami, H., Kawase, T., Hirose, K., Mitani, A., Sumiyoshi, W., Kinoshita, T., Abe, J., Yasunaga, M., Matsumura, Y., and Ito, Y. (2019) Characterization of Antibody Products Obtained through Enzymatic and Nonenzymatic Glycosylation Reactions with a Glycan Oxazoline and Preparation of a Homogeneous Antibody-Drug Conjugate via Fc N-Glycan. Bioconjug Chem 30, 1343-1355.
Mindt, T. L., Jungi, V., Wyss, S., Friedli, A., Pla, G., Novak-Hofer, I., Grunberg, J., and Schibli, R. (2008) Modification of different IgG1 antibodies via glutamine and lysine using bacterial and human tissue transglutaminase. Bioconjug Chem 19, 271-8.
Moulton, K. R.; Sadiki, A.; Koleva, B. N.; Ombelets, L. J.; Tran, T. H.; Liu, S.; Wang, B.; Chen, H.; Micheloni, E.; Beuning, P. J.; O'Doherty, G. A.; Zhou, Z. S., Site-Specific Reversible
Park, J.; Chariou, P. L.; Steinmetz, N. F., Site-Specific Antibody Conjugation Strategy to Functionalize Virus-Based Nanoparticles. Bioconjug Chem 2020, 31 (5), 1408-1416.
Protein and Peptide Modification: Transglutaminase-Catalyzed Glutamine Conjugation and Bioorthogonal Light-Mediated Removal. Bioconjug Chem 2019, 30 (6), 1617-21.
Qian, J., Liu, T., Yang, L., Daus, A., Crowley, R., and Zhou, Q. (2007) Structural characterization of N-linked oligosaccharides on monoclonal antibody cetuximab by the combination of orthogonal matrix-assisted laser desorption/ionization hybrid quadrupole-quadrupole time-of-flight tandem mass spectrometry and sequential enzymatic digestion. Anal Biochem 364, 8-18.
Sadiki A., Vaidya S. R., Abdollahi M., Bhardwaj G., Dolan M. E., Turna H., Arora V., Sanjeev A., Robinson T. D., Koid A., Amin A., and Zhou Z. S. Site-specific Conjugation of Native Antibody. Antibody Therapeutics, 2020, 3, 271-284.
Scallon, B. J., Moore, M. A., Trinh, H., Knight, D. M., and Ghrayeb, J. (1995) Chimeric anti-TNF-alpha monoclonal antibody cA2 binds recombinant transmembrane TNF-alpha and activates immune effector functions. Cytokine 7, 251-9.
Schneider, H., Deweid, L., Avrutina, O., and Kolmar, H. (2020) Recent progress in transglutaminase-mediated assembly of antibody-drug conjugates. Anal Biochem 595, 113615.
Sjogren, J., Struwe, W. B., Cosgrave, E. F., Rudd, P. M., Stervander, M., Allhorn, M., Hollands, A., Nizet, V., and Collin, M. (2013) EndoS2 is a unique and conserved enzyme of serotype M49 group A Streptococcus that hydrolyses N-linked glycans on IgG and alpha1-acid glycoprotein. Biochem J 455, 107-18.
Sojar, H. T. and Bahl, O. P. (1987) A Chemical Method for the Deglycosylation of Proteins. Arch. Biochem. Biophys. 259, 52-57.
Strop, P., Tran, T. T., Dorywalska, M., Delaria, K., Dushin, R., Wong, O. K., Ho, W. H., Zhou, D., Wu, A., Kraynov, E., Aschenbrenner, L., Han, B., O'Donnell, C. J., Pons, J., Rajpal, A., Shelton, D. L., and Liu, S. H. (2016) RN927C, a Site-Specific Trop-2 Antibody-Drug Conjugate (ADC) with Enhanced Stability, Is Highly Efficacious in Preclinical Solid Tumor Models. Mol Cancer Ther 15, 2698-2708.
Tsai, T. I., Li, S. T., Liu, C. P., Chen, K. Y., Shivatare, S. S., Lin, C. W., Liao, S. F., Lin, C. W., Hsu, T. L., Wu, Y. T., Tsai, M. H., Lai, M. Y., Lin, N. H., Wu, C. Y., and Wong, C. H. (2017) An Effective Bacterial Fucosidase for Glycoprotein Remodeling. ACS Chem Biol 12, 63-72.

1. A method for making a site-specific modification of a glycoprotein, the method comprising the steps of:
(a) trimming a glycan linked to a glycosylation site amino acid residue of the glycoprotein, whereby a core of the glycan remains linked to the glycosylation site amino acid residue, and whereby a modification site amino acid residue of the glycoprotein becomes accessible for a reaction; and
(b) conjugating a reagent to the modification site amino acid residue made accessible in step (a).

2. The method of claim 1, wherein the glycosylation site amino acid residue is an asparagine residue, and the glycan is an N-glycan linked to a side chain of the asparagine residue.

3. The method of claim 1, wherein the modification site amino acid residue made accessible is a glutamine residue.

4. The method of claim 3, wherein said glutamine residue is a naturally occurring glutamine residue or a non-naturally occurring glutamine residue for said glycoprotein.

5. The method of claim 3, wherein the glycosylation site amino acid residue is an asparagine residue and the glutamine residue is adjacent to the asparagine residue or two residues away from the asparagine residue.

6. The method of claim 1, wherein the conjugation is catalyzed by a transglutaminase enzyme and the reagent is an amine-containing reagent.

7. The method of claim 6, wherein the amine-containing reagent becomes covalently linked through its amine function to a side chain of the glutamine residue.

8. The method of claim 6, wherein the transglutaminase enzyme is a naturally occurring transglutaminase, mutated transglutaminase, engineered transglutaminase, recombinant transglutaminase, micro-organism transglutaminase, mammalian transglutaminase, or protein-glutamine γ-glutamyltransferase E.C. 2.3.2.13.

9. The method of claim 1, wherein the glycoprotein comprises an N-linked glycosylation site having an amino acid sequence selected from the group consisting of SEQ ID NOS:1-17.

10. The method of claim 2, wherein the core of the N-glycan remaining after step (a) comprises at least one N-acetyl glucosamine residue.

11. The method of claim 1, wherein the glycosylation site amino acid residue is a serine residue or a threonine residue, and the glycan is an O-glycan linked to a side chain of the serine or threonine residue.

12. The method of claim 11, further comprising the step of, before or during step (a):
(a0) removing one or more sialic acid residues from the O-glycan using a sialidase.

13. The method of claim 1, wherein the trimming is performed using an endoglycosidase.

14. The method of claim 13, wherein the endoglycosidase is a naturally occurring, mutated, recombinant, or engineered endoglycosidase.

15. The method of claim 13, wherein the endoglycosidase is selected from the group consisting of Endo S, Endo S2, Endo H, Endo F1, Endo F2, Endo F3, Endo D, and Endo M

16. The method of claim 13, wherein the endoglycosidase mediates hydrolysis of an N,N′diacetyl chitobiose bond.

17. The method of claim 13, wherein the endoglycosidase is endo-α-N-acetylgalactosaminidase.

18. The method of claim 1, wherein the trimming is performed using a chemical method.

19. The method of claim 18 comprising incubation of the glycoprotein with trifluoromethanesulfonic acid.

20. The method of claim 1, wherein said glycosylation site amino acid residue is neutrally charged before performing the method, and wherein the amino acid residue remains neutrally charged after performing the method.

21. The method of claim 1, further comprising at least one of the following additional steps performed before, during, or after step (b):
transferring a galactose moiety comprising a chemical handle onto the trimmed core glycan using a β-1,4-galactosyltransferase;
transferring an unnatural glycan substrate onto the trimmed core glycan using a glycosynthase;
removing a fucose from the trimmed core glycan using a fucosidase.

22. The method of claim 21, wherein the glycan is an N-glycan, and the trimmed core of the N-glycan is a trimmed core N-glycan.

23. The method of claim 22, wherein a galactose moiety comprising a chemical handle is transferred onto the trimmed core N-glycan using a galactosyltransferase, and wherein the method further comprises the step of:
attaching a functional group to the chemical handle; wherein the functional group comprises a chromophore, fluorophore, affinity tag or targeting agent, chelator, radioisotope, dye or contrast agent, ultrasound agent, targeting moiety, polyethylene glycol, or polymer.

24. The method of claim 1, wherein in step (b) the reagent comprises an amine-containing reagent that becomes covalently linked via an isopeptidic bond between an amine group of the amine-containing reagent and an acyl group on a side chain of the at least one of said one or more modification site amino acid residues.

25. The method of claim 1, wherein the reagent is an amine-containing reagent comprising a clickable handle for click chemistry.

26. The method of claim 25, wherein the clickable handle comprises an azide functional group.

27. The method of claim 26, further comprising the step of:
attaching a moiety comprising a complementary clickable handle, wherein the complementary clickable handle is capable of forming a bond between the azide functional group and the complementary clickable handle.

28. The method of claim 27, wherein the complementary clickable handle comprises a strained alkyne functional group.

29. The method of claim 28, wherein the complementary clickable handle forms a bond with the azide functional group via a strain promoted azide-alkyne cycloaddition (SPAAC) reaction.

30. The method of claim 1, wherein the reagent or the moiety comprising a clickable handle comprises a targeting moiety, an imaging moiety, an immunomodulator, a gene delivery vehicle, a therapeutic agent, a diagnostic agent, or a polymer.

31. The method of claim 1, wherein the reagent is an amine-containing reagent comprising a stimulus-responsive linker selected from the group consisting of a photo-responsive linker, acid-cleavable linker, reducible linker, peptide or dipeptide linker, and a β-glucuronide linker.

32. The method of claim 1, wherein the glycoprotein is selected from the group consisting of antibodies or antibody fragments and glycosylated enzymes.

33. The method of claim 1, wherein the glycoprotein is a naturally occurring or non-naturally occurring antibody or antibody fragment.

34. The method of claim 1, wherein the glycoprotein has a biological activity which is preserved after said site-specific modification.

35. The method of claim 1, wherein a naturally occurring antibody is modified while retaining its primary sequence, core glycan, and binding specificity.

36. A modified glycoprotein produced by the method of claim 1.

37. A kit for site-specific modification of a glycoprotein, the kit comprising:
an endoglycosidase;
a transglutaminase; and
instructions for performing the method of claim 1.

38. The kit of claim 37, further comprising a galactosyltransferase or a glycosynthase.

39. The kit of claim 37, further comprising an amine-containing reagent.

40. The kit of claim 39, wherein the amine-containing reagent comprises a targeting moiety, an imaging moiety, an immunomodulator, a gene delivery vehicle, a therapeutic agent, a diagnostic agent, or a polymer.

41. The kit of claim 39, wherein the amine-containing reagent comprises an azide functional group that can undergo biorthagonal conjugation, such as inverse electron demand Diels-Alder (IEDDA) involving 1,2,4,5-tetrazine and an olefin.

42. The kit of claim 39, comprising a moiety comprising a strained alkyne functional group and instructions for performing a SPAAC reaction.

43. A method for modulating an effector function of an antibody, the method comprising the steps of:
(a) trimming a glycan linked to a glycosylation site amino acid residue of the antibody using an endoglycosidase, whereby a core of the glycan remains linked to the glycosylation site amino acid residue, and whereby the effector function of the antibody is reduced or abolished; and
(b) transferring a glycan substrate onto the core of the glycan using a glycosynthase or removing a fucose from the core of the glycan using a fucosidase, whereby the effector function is at least partially recovered.

44. The method of claim 43, wherein the endoglycosidase is Endo S2.

45. The method of claim 43 wherein the effector function is abolished after performing step (a).

46. The method of claim 43, wherein the effector function is binding of an Fc domain of the antibody to an Fcγ receptor on an immune cell.

47. The method of claim 43, wherein the effector function comprises antibody-dependent cellular cytotoxicity or complement-dependent cytotoxicity.

48. The method of claim 43, wherein the glycan substrate comprises oxazoline.

49. The method of claim 43, wherein the glycosylation site amino acid residue is an asparagine residue, and the glycan is an N-glycan linked to a side chain of the asparagine residue.

50. The method of claim 43, wherein the antibody comprises one or more glutamine residues, and
wherein in step (a) the one or more glutamine residues become accessible to a transglutaminase enzyme for a transglutaminase-catalyzed reaction between the one or more glutamine residues and an amine-containing reagent.

51. The method of claim 50, wherein said one or more glutamine residues are naturally occurring glutamine residues or non-naturally occurring glutamine residues for said antibody.

52. The method of claim 50, further comprising the step of, before, during, or after step (b):
(b1) conjugating the amine-containing reagent to the one or more accessible glutamine residues via the transglutaminase-catalyzed reaction, whereby the reagent becomes covalently linked through its amine function to a side chain of at least one of said one or more glutamine residues.

53. An antibody having a modulated effector function, the antibody made by the method of claim 43.

54. The modified glycoprotein of claim 36 that is suitable for use as an imaging agent.

55. The modified glycoprotein or the modulated antibody of claim 54, wherein the imaging is of a cell or of a tumor.

56. The modified glycoprotein of claim 36 that is suitable for use in a targeted therapy for treating a cancer, for use in a biological assay, for use in a diagnostic procedure, or for use in a method comprising immunotherapy.
